(19) United States
(12) Patent Application Publication
(10) Pub. No.: US 2018/0002381 A1
(43) Pub. Date: Jan. 4, 2018

(54) HYDROCARBON-STAPLED POLYPEPTIDES FOR ENHANCEMENT OF ENDOSOME-LYSOSOMAL DEGRADATION

(71) Applicant: The Hong Kong Polytechnic University, Hong Kong (CN)

(72) Inventors: Yanxiang ZHAO, Hong Kong (CN); Shuai WU, Hong Kong (CN); Wenchao YANG, Hong Kong (CN); Yunjiao HE, Hong Kong (CN); Xiaohua LI, Hong Kong (CN); Xianxiu QIU, Hong Kong (CN)

(21) Appl. No.: 15/636,999

(22) Filed: Jun. 29, 2017

Related U.S. Application Data
(60) Provisional application No. 62/355,883, filed on Jun. 29, 2016.

(51) Int. Cl. C07K 7/08; A61K 38/00

(52) U.S. Cl. CPC C07K 7/08; A61K 38/00

(57) ABSTRACT

The present invention relates to a Beclin1-UVRAG complex structure which reveals a tightly packed coiled coil assembly with Beclin 1 and UVRAG residues complementing each other to form a stable dimeric complex. This potent physical interaction is critical for UVRAG-dependent EGFR degradation but less critical for autophagy. Targeting the Beclin coiled coil domain with rationally designed stapled peptides leads to enhanced autophagy activity and EGFR degradation in non-small cell lung cancer (NSCLC) cell lines, suggesting translational value for these compounds.

### CROSS-REFERENCE TO RELATED APPLICATIONS

This application claims the benefit of U.S. Provisional Application No. 62/355,883, filed Jun. 29, 2016. The entire contents and disclosures of the preceding application are incorporated by reference into this application.

Throughout this application, various publications are cited. The disclosures of these publications in their entireties are hereby incorporated by reference into this application to more fully describe the state of the art to which this invention pertains.

### FIELD OF THE INVENTION

This invention relates to designed peptide analogs that promote autophagy by specifically targeting the Beclin1-Vps34 complex.

### BACKGROUND OF THE INVENTION

UV irradiation resistance-associated gene (UVRAG) has been implicated in diverse cellular processes including autophagy, endocytic trafficking and chromosome maintenance. UVRAG was first identified from a cDNA library screening for its ability to complement partially the ultraviolet sensitivity of a xeroderma pigmentosum cell line (Perelman et al., 1997). UVRAG was recently found to be a key regulator of the Class III Phosphotidylinositol 3-Kinase (PI3K) complex, a critical component of the molecular machinery of autophagy consisting of the scaffolding protein Beclin 1 and the lipid kinase VPS34 as core members. Through potent and specific interaction with Beclin 1, UVRAG can lead to the formation of UVRAG-containing Beclin1-VPS34 complex with enhanced lipid kinase activity to direct VPS34-related cellular processes such as autophagy (Liang et al., 2006; Liang et al., 2007). UVRAG has also been found to associate with Class C Vps complex and coordinate endocytic trafficking (Liang et al., 2008a; Liang et al., 2008b). Furthermore UVRAG plays a role in maintaining structural integrity and proper segregation of chromosomes through its interactions with centrosome protein CEP63 and DNA-PK that is involved in homologous end joining (Zhao et al., 2012).

UVRAG contains two well predicted functional domains based on sequence alignment. The N-terminal C2 domain is regarded to associate with membrane and be involved in autophagy and endosomal trafficking (Liang et al., 2006). The coiled coil (CC) domain is critical for binding to Beclin 1, the essential autophagy scaffolding protein, to form the autophagy-promoting UVRAG-containing Beclin 1-VPS34 complex (Liang et al., 2006). In addition to these two domains, the N-terminal proline-rich sequence of UVRAG interacts with the SH3 domain of Bif-1 and probably enables Bif-1 to promote autophagosome formation through its membrane-curving BAR domain (Takahashi et al., 2007; Takahashi et al., 2009). The region between the coiled coil domain and the C-terminal PEST-like sequence is involved in interaction with Class C Vps complex, CEP63 and DNA-PK (Liang et al., 2008a; Zhao et al., 2012).

No structural information at atomic resolution is currently available regarding UVRAG, and the molecular mechanism of how the individual functional domains of UVRAG associate with their respective binding partners to regulate diverse cellular processes of autophagy, endocytic trafficking and chromosomal segregation is not well understood.

The interaction between Beclin 1 and two central autophagy regulators Atg14L and UVRAG is mediated through their respective coiled coil domains (Liang et al., 2006; Matsunaga et al., 2009; Zhong et al., 2009). The structure of the Beclin 1 coiled coil domain was determined previously, which forms a metastable antiparallel coiled coil structure due to several charged or polar residues that destabilize an otherwise hydrophobic dimer interface (Li et al., 2012a). This metastability is found to be important for Beclin 1's interaction with Atg14L or UVRAG because it enables the homodimeric Beclin 1 to readily dissociate and form heterodimeric assembly with Atg14L and UVRAG (Li et al., 2012a). Mutations within the Beclin 1 coiled coil domain that render it monomeric retains its binding to Atg14L or UVRAG and facilitates normal autophagy induction; while mutations that stabilize the Beclin 1 homodimer weaken or abolish its interaction with Atg14L and lead to impaired autophagosome formation (Li et al., 2012a; Li et al., 2012b).

The mammalian Class III phosphatidylinositol 3-kinase (PI3KC3) complex, also termed the Beclin1-Vps34 complex, is a dynamic multi-protein assembly that plays critical roles in membrane-mediated intracellular transportation processes such as autophagy, endocytic trafficking and phagocytosis. Core members of this complex include the lipid kinase Vps34 that serves as the major producer of phosphatidylinositol 3-phosphate (PI3P) lipids; a serine/threonine kinase Vps15 stably associated with Vps34, the scaffolding molecule Beclin1 and either Atg14L or UVRAG as the Beclin1-binding partner. The Atg14L-containing form is termed Beclin1-Atg14L complex and mainly involved in early-stage autophagy induction because Atg14L is responsible for directing Beclin1-Atg14L complex to ER sites to promote autophagosome biogenesis. The UVRAG-containing form, on the other hand, is termed Beclin1-UVRAG complex and plays critical roles in late-stage autophagy execution and degradative endocytic trafficking. In addition to these core molecules, many regulators such as Ambra1, Bcl-2, NRBF2 and Rubicon can associate with the Beclin1-Vps34 complex in dynamic and context-dependent manner to exert modulatory effect on the Vps34 kinase activity. The molecular mechanism of such regulation, particularly whether these diverse molecules share a common theme of modulating the structural and thus biochemical properties of the Beclin1-Vps34 complex, is not well understood.

The recent electron microscope (EM) structures of Beclin1-Atg14L complex and Beclin1-UVRAG complex reconstructed at about 27 Å resolution revealed a V-shaped architecture that is highly dynamic. In particular, the catalytic domain of Vps34 is largely unhinged from the main body of the complex and undergoes long-range swinging motions. Crystal structure of the yeast homolog of Beclin1-UVRAG complex solved at 4.4 Å resolution showed a similar Y architecture with Vps34 and Vps15 forming the catalytic arm while Atg30 and Atg38 (homologs of Beclin1 and UVRAG) forming the regulatory arm. Structure-based functional studies confirm that full catalytic activity requires both arms of the Y shape to be properly associated with target membrane. Furthermore, hydrogen deuteron exchange (HDX) analysis revealed that the event of membrane binding induces conformational changes within certain regions of the Beclin1-Vps34 complex that are compatible with global “opening” and “closing” motions. A model derived from these studies proposes that, for Beclin1-Atg14L complex and Beclin1-UVRAG complex, conformational coordination between the Beclin1-Atg14L/UVRAG regulatory arm and the Vps15-Vps34 catalytic arm determines the “aperture” of the Y-shape to fit onto different membrane targets. Specifically, Beclin1-Atg14L complex and Beclin1-UVRAG complex both achieve high activity on nascent autophagic membranes by “closing” its two arms to fit their high-curvature surfaces. However, only Beclin1-UVRAG complex can adapt to low-curvature membranes like endosomes by “opening” its two arms more apart.

A prominent feature in both the EM structure of Beclin1-Atg14L complex and the crystal structure of Beclin1-UVRAG complex is the long stretch of coiled coil that runs through the regulatory arm within the Y architecture. This region corresponds to the interaction site where Atg14L and UVRAG bind to Beclin1 in mutually exclusive manner via their respective coiled coil domains. Previously it has been determined that the Beclin1 coiled coil domain forms a metastable antiparallel coiled coil assembly due to several charged or polar residues that destabilize an otherwise hydrophobic dimer interface.

Previous biochemical studies also reveal that Atg14L or UVRAG forms heterodimeric complex with Beclin1 but it is not known how the “imperfect” features within the coiled coil region of Beclin1 can facilitate its specific interaction with Atg14L and UVRAG. The potency of the Atg14L/UVRAG-Beclin1 interaction is also not clear but may bear functional significance because it may influence the structural flexibility of the Beclin1-Vps34 complex, particularly for the “closing” and “opening” motions proposed by the prevailing model. At present, atomic structural model of the Atg14L/UVRAG-Beclin1 interaction cannot be extracted from the EM and crystal structures of Beclin1-Atg14L complex and Beclin1-UVRAG complex due to their limited resolutions. Hence, there is a need to have further structural and functional studies to examine the structure and functions of the Beclin1-UVRAG complex.

### SUMMARY OF THE INVENTION

In the present invention, the crystal structure of the Beclin1-UVRAG coiled coil complex and structure-based analysis to delineate the molecular determinants that drive the formation of a stable Beclin1-UVRAG complex is studied. The functional significance of the potent Beclin1-UVRAG interaction in mediating Vps34-dependent autophagy and endocytic trafficking is also investigated. Lastly, the structure of the Beclin1-UVRAG complex can be used to guide the design of hydrocarbon-stapled peptides that specifically target Beclin1 coiled coil domain and promote Vps34-dependent autophagy and lysosomal degradation of epithelial growth factor receptor (EGFR).

The present invention discloses a hydrocarbon-stapled polypeptide designed to target a polypeptide comprising amino acid residues 231-245 of rat Beclin 1 (SEQ ID No.: 15:YSEFKRQQLELDDEL), or amino acids 233-247 of human Beclin 1 (SEQ ID No.: 16: YSEFKRQQLELDDEL), wherein the hydrocarbon-stapled polypeptide comprises an amino acid sequence that is at least 85% identical to amino acid residues 191-205 of rat Beclin 1 (SEQ ID No.: 17: RLIQELEDVEKNRKV), or amino acids 193-207 of human Beclin 1 (SEQ ID No.: 18: RLIQELEDVEKNRKI).

The present invention discloses a pharmaceutical composition comprising the hydrocarbon-stapled polypeptide of the present invention.

The present invention also discloses a method of enhancing autophagy or endocytic trafficking, comprising the step of contacting a population of cells with the hydrocarbon-stapled polypeptide of the present invention, thereby enhancing lysosomal degradation of one or more target proteins.

The present invention further discloses a method of inhibiting cancer cell growth, comprising administering the hydrocarbon-stapled polypeptide of the present invention to a subject in need thereof.

### BRIEF DESCRIPTION OF THE DRAWINGS

FIG. 1A shows the constructs designed to map the core region of Beclin coiled coil domain responsible for UVRAG interaction. Four constructs of Beclin 1 coiled coil 1-4 (CC1-4, SEQ ID No.: 19-22), each consisting of 7 heptad repeats, are engineered to scan through the entire Beclin coiled coil domain. Each heptad repeat is represented by a segment with gradient color from N- to C-terminal. (CC=SEQ ID No.: 23)

FIG. 1B shows Isothermal Titration Calorimetry (ITC) profiles used to measure the binding affinity of Beclin 1 CC1-4 (SEQ ID No.: 19-22) for UVRAG interaction. CC1 (SEQ ID No.: 19) binds to UVRAG coiled coil domain the strongest with Kd comparable to that of wild-type.

FIG. 1C shows the linked Beclin 1-UVRAG construct used in structural studies.

FIG. 1D shows the parallel dimeric coiled coil structure of the Beclin 1-UVRAG complex.

FIG. 1E shows helical wheel presentation of the Beclin1-UVRAG coiled coil dimer interface.

FIG. 2A shows a comparison of the coiled coil interface of Beclin 1 homodimer and the Beclin 1-UVRAG heterodimer. The a-d′ pairings observed in Beclin1 homodimer and the a-a′/d-d′ pairings observed in Beclin 1-UVRAG complex are aligned according to Beclin 1 sequence. The residues at position a of the heptad repeat motif are marked with a box while residues at position d are not marked with box. Arrows indicate the energetically favorable pairings at the interface. Arrows (*) mark the strongly hydrophobic “leucine zippers”; Arrows (#) mark the moderately hydrophobic pairings and arrows (̂) mark the stabilizing pairings only observed in Beclin1-UVRAG complex.

FIG. 2B shows ITC profile to measure the interaction between Beclin1 and UVRAG mutants after the leucine residues critical for forming the hydrophobic coiled coil interface are replaced by glutamate. WT: wild-type. 1E to 6E denote the single to multiple Leu-to-Glu mutations on UVRAG coiled coil domain.

FIG. 2C shows co-immunoprecipitation results to characterize the association between Beclin1 and UVRAG Leu-to-Glu mutants in vivo. FLAG-tagged UVRAG constructs were transfected into HEK293T cells. Their interaction with endogenous Beclin1 was probed using anti-FLAG@M2 magnetic beads for immunoprecipitation, followed by immunoblotting (IB) using anti-Beclin1 antibody. Results were repeated under both normal fed (−) or EBSS starvation (+) conditions.

FIG. 2D shows competitive co-immunoprecipitation experiments to compare the in vivo potency of Beclin1 association between UVRAG mutants and Atg14L. FLAG-tagged UVRAG constructs and GFP-tagged Atg14L were co-transfected into HEK293T cells. The association between UVRAG mutants and endogenous Beclin1 in the presence of over-expressed Atg14L as competitor was probed using anti-FLAG@M2 magnetic beads for immunoprecipitation, followed by immunoblotting (IB) using anti-Beclin1 antibody.

FIG. 2E shows competitive co-immunoprecipitation experiments similar to FIG. 2D. The tags for UVRAG and Atg14L were swapped so that FLAG-tagged Atg14L and GFP-tagged UVRAG mutants were co-transfected into HEK293T cells. The association between Atg14L and endogenous Beclin1 in the presence of over-expressed UVRAG mutants as competitor was probed in the same manner as FIG. 2D.

FIG. 3A shows representative confocal fluorescent images of HeLa cells stably expressing GFP-LC3 after transfection of mCherry-tagged UVRAG wild-type (WT) and 6E mutant construct. The pattern of LC3 puncta formation was little affected by over-expression of either WT or 6E.

FIG. 3B shows western blots of autophagy marker LC3 in HeLa cells after co-transfection with FLAG-tagged UVRAG constructs either in the absence (−) or presence (+) of hydroxychloroquine (CQ). The profile of LC3 lipidation, as assessed by the ratio of LC3-I/LC3-II, is nearly the same for all UVRAG constructs.

FIG. 3C shows western blots of autophagy marker p62 and LC3 in HEK293T cells after transfection with FLAG-tagged UVRAG wild type (WT) or UVRAG mutant constructs (1E, 2E, 5E and 6E) under amino acids starvation condition. No difference in terms of p62 level and LC3 lipidation was observed between the wild type and the mutant constructs either in the absence (−) or presence (+) of hydroxychloroquine (CQ).

FIG. 3D shows the impact of UVRAG Leu-to-Glu mutations on EGFR degradation profile in HEK293T cells. After transfection with UVRAG constructs, HEK293T cells were starved overnight and treated with EGF. EGFR level in cell lysate was analyzed by western blots at specific time points over a period of 120 minutes. Over-expression of wild-type, 1E and 2E UVRAG constructs lead to enhanced EGFR degradation while 5E and 6E fail to do so.

FIG. 3E shows EGFR degradation profile in A549 non-small cell lung cancer cells. The time frame for EGFR degradation in A549 cells is much prolonged (5 hours) as compared to that in HEK293T (˜2 hours). Nonetheless similar results were obtained. Over-expression of wild-type, 1E and 2E constructs lead to enhanced EGFR degradation while 5E and 6E fail to do so.

FIG. 3F shows the quantification of results as shown in FIG. 3E from three independent repeated experiments.

FIG. 4A shows the design principle of Beclin1-specific α-helical stapled peptides. The coiled coil domains of Beclin1 and UVRAG are drawn in relative scale to demonstrate the hydrophobic interface formed between the N-terminal half of Beclin1 coiled coil domain and UVRAG. The stapled peptide is shown as a short ribbon. The spheres on the ribbon represent the chemically engineered staples to stabilize the α-helical structure. The two Ys mark Beclin1 residue Y227 and Y231, which correspond to the EGFR-phosphorylated Y229 and Y233 in human Beclin 1. The stapled peptide is designed to bind to the C-terminal half of Beclin1 coiled coil region starting from around Y227 and Y231.

FIG. 4B shows ITC profile to show that Beclin1 with mutation in the C-terminal region of its coiled coil domain (mBeclin1, monomeric Beclin 1) binds to UVRAG coiled coil (Kd=10 nM) stronger than wild-type (Kd=0.24 μM). mBeclin 1 also binds strongly to Atg4L coiled coil (SEQ ID No.: 25) (Kd=0.5 μM).

FIG. 4C shows a model of a computationally designed stapled peptide SP1 (SEQ ID No.: 1) binding to the C-terminal region of Beclin1 coiled coil domain. The bracket highlights the hydrocarbon staple. The residues are numbered according to Beclin1 sequence.

FIG. 4D shows circular dichroism spectra of stapled peptide SP4 (SEQ ID No.: 4) and its unstapled version P4. SP4 (SEQ ID No.: 4) shows significantly higher α-helical content.

FIG. 4E shows computational modeling to optimize the amino acid sequence of designed stapled peptides. The residues deemed critical for Beclin1 binding are marked with “*” and remain unchanged. Residues subject to computational mutation are marked with “̂” Molecular dynamics (MD) simulations were conducted to evaluate the binding modes of the designed peptides, and the binding energies were computed using the force field-based MM-GB/SA method. Three highlighted candidates. SP4 (SEQ ID No.: 4), SP9 (SEQ ID No.: 9) and SP12 (SEQ ID No.: 12) show significantly improved binding as compared to SP1 (SEQ ID No.: 1).

FIG. 4F shows SP4 (SEQ ID No.: 4) binds to Beclin1 coiled coil domain (Kd=2 μM) as confirmed by ITC measurements.

FIG. 4G shows SP4 (SEQ ID No.: 4) induces dimer-to-monomer transition as shown by dynamic light scattering measurements. Beclin1 coiled coil domain mixed with SP4 (SEQ ID No.: 4) was passed through size exclusion chromatography and its time-dependent profile for dynamic light scattering (̂) and UV absorbance (#) are plotted. The oligomeric state of the Beclin 1 molecule is inferred from the molecular weight estimated from the light scattering profile.

FIG. 4H shows representative confocal fluorescence images of Rhodamine-labeled SP4 (SEQ ID No.: 4) colocalizes with GFP-tagged Beclin1 in A549 cell. A549 cells transiently expressing GFP-Beclin1 were treated with 20 μM Rhodamine-SP4 (SEQ ID No.: 4) for 30 minutes and observed under a confocal microscope.

FIG. 5A shows representative confocal fluorescence images of HeLa cells stably expressing GFP-LC3 after treatment with empty vehicle (control), Tat-tagged scrambled peptide (SC4, SEQ ID No.: 14: Ac-RALRIQSKEELRD-NH2) and Tat-tagged SP4 stapled peptide (SP4, SEQ ID No.: 4). Experiments were done both in the absence (−) or presence (+) of chloroquine.

FIG. 5B shows histogram to show quantification of the results from FIG. 5A. Error bars represent ±s.e.m of triplicate samples. Vehicle: empty vector as control. ***P, 0.05. t-test.

FIG. 5C shows western blots to assess the LC3 lipidation profile in HEK293T cell after treatment with scrambled or stapled peptide at both low dosage (L, 10 μM) and high dosage (H, 20 μM) in the absence of chloroquine (CQ, 50 μM).

FIG. 5D shows western blots to assess the p62 level and LC3 lipidation profile in HEK293T cell after treatment with scrambled or stapled peptide at both low dosage (L, 10 μM) and high dosage (H, 201 μM) in the presence (+) of chloroquine (CQ, 50 μM).

FIG. 5E shows EGFR degradation profile in HEK293T cells after treatment with scrambled or stapled peptides.

FIG. 5F shows time-dependent plot of FIG. 5E after three independent experiments.

FIG. 5G shows EGFR degradation profile in A549 cells after treatment with scrambled or stapled peptides.

FIG. 5H shows EGFR degradation profile in H1975 cells after treatment with scrambled or stapled peptides.

FIG. 5I shows time-dependent plot of FIG. 5H after three independent experiments.

FIG. 6 shows potent Beclin1-UVRAG interaction via their respective coiled coil domain favoring the formation of UVRAG-containing Beclin1-Vps34 complex to promote endocytic degradation of EGFR at an early stage upstream of the late-endosome stage promoted by UVRAG via interaction with Class C Vps complex. Rationally designed stapled peptides (star shapes) can disrupt the metastable Beclin1 homodimer and assist the Beclin1-Atg14L/UVRAG interaction to concomitantly promote autophagy and lysosomal degradation of EGFR.

FIG. 7A shows ITC profile of UVRAG (228-298) (SEQ ID No.: 27) binding to wild type form of Beclin1 coiled coil domain.

FIG. 7B shows ITC profile of UVRAG (228-298) (SEQ ID No.: 27) binding to monomeric form of Beclin1 coiled coil domain. The monomeric form of Beclin1 coiled coil domain was constructed with L182A mutation (SEQ ID No.: 33).

FIG. 8 shows additional favorable pairings at the Beclin1-UVRAG interface. The extra “leucine zipper” pair (L210d-L264d′) and the electrostatically favorable pairing (R203d-E269d′) at the Beclin1-UVRAG heterodimer interface are depicted. Each residue is illustrated in ball-and-stick model. The packing is illustrated by van der Waals spheres depicting the side-chain atoms.

### DETAILED DESCRIPTION OF THE INVENTION

The present invention discloses formation of a more stable heterodimeric coiled coil assembly of Beclin1 and UVRAG. The present invention further relates to enhanced VPS lipid kinase activity and autophagy induction by the stable Beclin 1-UVRAG complex.

The present invention discloses the feature of the Beclin1 coiled coil domain's dimer interface and suggests its role in modulating formation of multiple distinct Beclin 1-VPS34 complexes that play essential roles in controlling various membrane trafficking pathways.

The present invention discloses how Beclin 1 and UVRAG form a more stable heterodimeric coiled coil assembly as compared to Beclin 1 homodimer and how this stable Beclin 1-UVRAG complex leads to enhanced VPS lipid kinase activity and autophagy induction.

The present invention also involves a Beclin1-UVRAG interface which is significantly stabilized by hydrophobic pairings and complementary interactions.

The present invention discloses a parallel coiled coil assembly revealing a Beclin1-UVRAG complex structure.

The present invention discloses potent Beclin1-UVRAG interaction via coiled coil domains which is required to promote UVRAG-dependent endosome-lysosomal degradation of EGFR. Furthermore, structure-based rational design of Beclin1-targeting stapled peptides are investigated. The present invention further discloses rationally designed stapled peptides that can promote autophagy and enhance EGFR degradation.

In one embodiment, the sequence of the peptide can be computationally optimized to achieve specific Beclin1 interaction. In one embodiment, hydrocarbon staples are designed to stabilize the peptide structure. In another embodiment, future modification or improvements of the stapled peptide can be done by improving the potency of the designed peptides by, for example, varying the amino acid composition or adding functional groups.

In one embodiment, Beclin1-specific stapled peptides that promotes autophagy and enhances lysosomal degradation of EGFR were designed.

In some embodiments, the peptides of the present invention can be used for anti-EGFR therapy. In a further embodiment, the peptides designed by the present invention can be used to target EGFR degradation by enhancing the Beclin1-UVRAG interaction. In one embodiment, the peptides designed by the present invention help to enhance EGFR degradation so as to reduce EGFR signaling and inhibit cell proliferation. In one embodiment, the peptides designed by the present invention can be used in anti-cancer therapy for EGFR-driven tumor types like non-small cell lung cancer (NSCLC) and breast cancer. In another embodiment, the present invention serves as orthogonal approach to existing NSCLC treatment regiments. In one embodiment, the peptides of the present invention can be used for treatment of neurodegenerative diseases where autophagy enhancement would be beneficial.

The present invention discloses a hydrocarbon-stapled polypeptide designed to target a polypeptide comprising amino acid residues 231-245 of rat Beclin 1 (SEQ ID No.: 15), or amino acids 233-247 of human Beclin 1 (SEQ ID No.: 16), wherein the hydrocarbon-stapled polypeptide comprises an amino acid sequence that is at least 85% identical to amino acid residues 191-205 of rat Beclin 1 (SEQ ID No.: 17), or amino acids 193-207 of human Beclin 1 (SEQ ID No.: 18). In one embodiment, the hydrocarbon-stapled polypeptide comprises an amino acid sequence that is at least 90% identical to amino acid residues 191-205 of rat Beclin 1 (SEQ ID No.: 17), or amino acids 193-207 of human Beclin 1 (SEQ ID No.: 18). the hydrocarbon-stapled polypeptide comprises an amino acid sequence that is at least 95% identical to amino acid residues 191-205 of rat Beclin 1 (SEQ ID No.: 17), or amino acids 193-207 of human Beclin1 (SEQ ID No.: 18).

In one embodiment, the hydrocarbon-stapled polypeptide is about 10-40 amino acids in length. In one embodiment, the hydrocarbon-stapled polypeptide is 10-30 amino acids in length. In one embodiment, the hydrocarbon-stapled polypeptide is 10-20 amino acids in length.

In one embodiment, the hydrocarbon-stapled polypeptide comprises one or more α,α-disubstituted 5-carbon olefinic amino acids. In one embodiment, the hydrocarbon-stapled polypeptide comprises one or more α,α-disubstituted 8-carbon olefinic amino acids. In one embodiment, the hydrocarbon-stapled polypeptide comprises unnatural amino acids at position i and position i+7. In one embodiment, the hydrocarbon-stapled polypeptide comprises a stabilized alpha-helix.

In one embodiment, the hydrocarbon-stapled polypeptide has an affinity for the polypeptide comprising amino acid residues 231-245 of rat Beclin 1 (SEQ ID No.: 15), or amino acids 233-247 of human Beclin 1 (SEQ ID No.: 16), of at least 5 μM. In one embodiment, the hydrocarbon-stapled polypeptide has an affinity for the polypeptide comprising amino acid residues 231-245 of rat Beclin 1 (SEQ ID No.: 15), or amino acids 233-247 of human Beclin 1 (SEQ ID No.: 16), of at least 2 μM.

In one embodiment, the hydrocarbon-stapled polypeptide has the sequence of one of SEQ ID NO. 1-12.

The present invention discloses a pharmaceutical composition comprising the hydrocarbon-stapled polypeptide of the present invention. The pharmaceutical composition of the present invention further comprises one or more pharmaceutically acceptable excipients, vehicles or carriers. In one embodiment, the pharmaceutical composition is formulated in the form of a cream, gel, ointment, suppository, tablet, granule, injection, powder, solution, suspension, spray, patch or capsule. In one embodiment, the pharmaceutical composition is administered orally, nasally, aurally, ocularly, sublingually, buccally, systemically, transdermally, mucosally, via cerebral spinal fluid injection, vein injection, muscle injection, peritoneal injection, subcutaneous injection, or by inhalation.

The present invention also discloses a method of enhancing autophagy or endocytic trafficking, comprising the step of contacting a population of cells with the hydrocarbon-stapled polypeptide of the present invention, thereby enhancing lysosomal degradation of one or more target proteins. In one embodiment, the target protein is EGFR. In one embodiment, the cells treated with the hydrocarbon-stapled polypeptide have decreased EGFR-driven cell proliferation.

The present invention further discloses a method of inhibiting cancer cell growth, comprising administering the hydrocarbon-stapled polypeptide of the present invention to a subject in need thereof. In one embodiment, the subject is a vertebrate, a mammal or human. In one embodiment, the cancer cell growth comprises EGFR-driven cell proliferation. In one embodiment, the cancer cells are non-small cell lung cancer cells, breast cancer cells, colon cancer cells, ovarian cancer cells, carcinoma cells, sarcoma cells, lung cancer cells, fibrosarcoma cells, myosarcoma cells, liposarcoma cells, chondrosarcoma cells, osteogenic sarcoma cells, chordoma cells, angiosarcoma cells, endotheliosarcoma cells, lymphangiosarcoma cells, lymphangioendotheliosarcoma cells, synovioma cells, mesothelioma cells, Ewing's tumor cells, leiomyosarcoma cells, rhabdomyosarcoma cells, gastric cancer cells, esophageal cancer cells, rectal cancer cells, pancreatic cancer cells, prostate cancer cells, uterine cancer cells, head and neck cancer cells, skin cancer cells, brain cancer cells, squamous cell carcinoma, sebaceous gland carcinoma cells, papillary carcinoma cells, papillary adenocarcinoma cells, cystadenocarcinoma cells, medullary carcinoma cells, bronchogenic carcinoma cells, renal cell carcinoma cells, hepatoma cells, bile duct carcinoma cells, choriocarcinoma cells, seminoma cells, embryonal carcinoma cells, Wilm's tumor cells, cervical cancer cells, testicular cancer cells, small cell lung carcinoma cells, bladder carcinoma cells, epithelial carcinoma cells, glioma cells, astrocytoma cells, medulloblastoma cells, craniopharyngioma cells, ependymoma cells, pinealoma cells, hemangioblastoma cells, acoustic neuroma cells, oligodendroglioma cells, meningioma cells, melanoma cells, neuroblastoma cells, retinoblastoma cells, T-cells or natural killer cells of leukemia, lymphoma cells, or Kaposi's sarcoma cells.

The invention will be better understood by reference to the Experimental Details which follow, but those skilled in the art will readily appreciate that the specific experiments are provided only for illustrative purpose, and are not meant to limit the invention scope as described herein, which is defined by the claims following thereafter.

It is to be noted that the transitional term “comprising”, which is synonymous with “including”, “containing” or “characterized by”, is inclusive or open-ended, and does not exclude additional, un-recited elements or method steps.

### Example 1

### Optimerization and Performance of Stapled Peptides

This example shows that the rationally optimized stapled peptide SP4 (SEQ ID No.: 4) can promote autophagy activity and enhance lysosomal degradation of EGFR in a Beclin1-dependent manner in multiple cell lines.

### 1. Reagents

Chloroquine (CQ; Sigma-Aldrich). Epidermal Growth Factor (EGF; Invitrogen), anti-(3-actin antibody (Santa Cruz Biotechnology), anti-Beclin1 antibody (Santa Cruz Biotechnology), anti-Flag antibody (Sigma-Aldrich), anti-Flag M2 Magnetic Beads (Sigma-Aldrich), protein A/G PLUS agarose beads (Santa Cruz Biotechnology), anti-GFP antibody (Roche), anti-LC3 antibody (Abnova), anti-p62 antibody (Abnova), Anti-Mouse IgG-HRP (Sigma-Aldrich), Anti-Rabbit IgG-HRP (Sigma-Aldrich), Lipofectamine 2000 (Invitrogen), Protease inhibitor cocktail (Roche Diagnostics), trypsin (Invitrogen), isopropyl-β-D-thiogalactopyranoside (IPTG; Sigma-Aldrich), PVDF membrane (Millipore), Fluorescence mounting medium (Calbiochem).

### 2. Protein Expression and Purification

The various fragments of UVRAG coiled coil domain were amplified by PCR using Mus musculus pCMV-UVRAG-FL as template and subcloned into modified pET-32a vector containing the human rhinovirus 3C protease cleavage site and thioredoxin-6×His fusion. The linked Beclin1-UVRAG coiled coil domain was constructed by inserting a “(Gly-Ser)5” segment between Beclin1 coiled coil fragment (174-223) and UVRAG coiled coil fragment (228-276) (SEQ ID No.: 26) and subsequently cloned into the same vector. All protein constructs were expressed in Escherichia coli BL21 (DE3) cells at 30° C. after induction by isopropyl-3-d-thiogalactopyranoside (IPTG) and purified by affinity chromatography (HisTrap HP, GE Healthcare). The fused tag was removed by 3C cleavage and the untagged protein was further purified by size-exclusion chromatography (Superdex 75, GE Healthcare).

### 3. Crystallization and Structure Determination

Crystals of Beclin1-UVRAG linked construct were grown at 16° C. by the hanging drop vapor diffusion method mixing 1 μl of Beclin1-UVRAG protein at 20 mg/ml with 1 μl of reservoir solution containing 3.0 M NaCl and 100 mM Citric acid buffer (pH 3.5). The Au derivative was obtained by soaking crystals in reservoir solution containing 5 mM KAu(CN)2 for about 10 seconds. The crystals were cryoprotected with 20% ethylene glycol prior to being mounted onto x538 ray source. All data sets were collected at beamline BL17U1 at Shanghai Synchrotron Radiation Facility (SSRF) in Shanghai, P.R.China All data sets were processed with the HKL3000 package29 and converted to CCP4 format for structure determination. The Au sites were located and refined using SOLVE31 and further refined using MLPHARE and DM32 modules in CCP4 to build an interpretable electron density map. The structure was built manually using COOT and the final structure was refined using REFMAC module in CCP4. Statistics were summarized in Table 1. The coordinates of Beclin1-UVRAG complex has been deposited to Protein Data Bank (PDB ID 5GKL). The structure figures were prepared using the CCP4 mg package in CCP4.

TABLE 1
Data collection, phasing and refinement statistics (SIRAS)

| | Native | | KAU(CN)₂ | |
|---|---|---|---|---|
| Data collection | | | | |
| Space group | P6122 | | P6122 | |
| Cell dimensions | | | | |
| a, b, c (Å) | 45.11, 45.11, 161.52 | | 45.16, 45.16, 161.94 | |
| α, β, γ (°) | 90, 90, 120 | | 90, 90, 120 | |
| Resolution (Å) | 50.00-1.80 | (1.86-1.80) | 50.00-1.84 | (1.95-1.84) |
| R_{sym} or R_{merge} | 7.0% | (16.5%) | 8.2% | (16.9%) |
| I/σI | 41.7 | (22.6) | 18.8 | (15.5) |
| Completeness (%) | 100 | (100) | 100 | (100) |
| Redundancy | 20.1 | (20.7) | 13.4 | (12.1) |

| | |
|---|---|
| Refinement | |
| Resolution (Å) | 37.97-1.90 |
| No. reflections | 7514 |
| R_{work}/R_{free} | 19.5%/24.4% |
| No. atoms | |
| Protein | 841 |
| Ligand/ion | N/A |
| Water | 164 |
| B-factors | |
| Protein | 20.83 |
| Ligand/ion | N/A |
| Water | 30.05 |
| R.m.s deviations | |
| Bond lengths (Å) | 0.018 |
| Bond angles (°) | 1.812 |

### 4. Isothermal Titration Calorimetry

Isothermal Titration Calorimetry was performed using an iTC200 microcalorimeter (MicroCal Inc.). Samples were dialyzed into 50 mM Tris, pH 8.0, and 150 mM NaCl. For Beclin 1-UVRAG interactions the injection syringe was loaded with 40 μl of Beclin 1 sample and the cell was loaded with 220 μl of UVRAG sample. Typically, titrations consisted of 20 injections of 2 μl, with 200-s equilibration between injections. The data were analyzed using Origin 7.0.

### 5. Static Light Scattering

Static light scattering was performed on Wyatt Dawn 8+(Wyatt Technology) that is connected to an AKTA FPLC system (GE Healthcare). The AKTA system was equipped with a size exclusion column (Superdex 200 10/30 GL, GE Healthcare) and equilibrated with at least one column-volume of Tris buffer until the light scattering signal became stable. Protein samples were centrifuged to remove any bubbles and particles before being loaded onto the system at the flow rate of 0.5 mL/min. UV and light scattering profiles were plotted and analysed by software ASTRA.

### 6. Plasmid Constructs for Cell-Based Studies

Full length Mus musculus UVRAG wild type (SEQ ID No.: 28), 1E (L246E) (SEQ ID No.: 29), 2E (L246E/L250E) (SEQ ID No.: 30), 5E(L232E/L239E/L246E/L250E/L264E) (SEQ ID No.: 31), and 6E (L232E/L239E/L246E/L250E/L264E/L271E) (SEQ ID No.: 32) were cloned into BamHI and XhoI sites of pcDNA3.1 Flag vector, HindIII and BamHI sites of pEGFP N3 vector and HindIII and BamHI sites of pmCherry Ni vector. Full length Mus musculus Atg14L was cloned into EcoRI and BamHI sites of pEGFP N3 vector following standard procedure.

### 7. Cell Culture

HEK293T, HeLa and A549 cell lines were cultured in Dulbecco's Modified Eagle's Medium (DMEM, Sigma) supplemented with 10% fetal bovine serum (FBS, Invitrogen). HeLa cell with stable expression of GFP-LC3 was a kind gift from Dr. Han Ming Shen's lab in National University of Singapore. All cell lines used in the experiments were mycoplasma detected negative by MycoAlert™ PLUS Mycoplasma Detection Kit (Lonza) before and during the experiment. Transient transfection was performed using Lipofectamine 2000 (Invitrogen) according to manufacturer's instruction.

### 8. Immunoblot Analysis

Transient DNA transfection was performed using Lipofectamine 2000 (Invitrogen). For Co-IP experiment to measure interaction between UVRAG and endogenous Beclin1, FLAG-tagged UVRAG plasmids were transfected into HEK293T cells. For Co-IP experiments to demonstrate competition between UVRAG and Atg14L for binding to endogenous Beclin1, equal amount of FLAG-tagged UVRAG mutant plasmids and GFP-tagged Atg14L plasmids or equal amounts of FLAG-tagged Atg14L plasmids and GFP-tagged UVRAG mutant plasmids were co-transfected into HEK293T cells. For immunoblotting assay of LC3-II, p62 and EGFR degradation, FLAG-tagged UVRAG mutant plasmids were transfected into HEK293T cells, HeLa cell stably expressing GFP-LC3 and A549 NSCLC cells respectively. Cells were lysed in IP buffer (25 mM HEPES PH 7.5, 10 mM MgCl₂, 150 mM NaCl, 1 mM EDTA.2Na, 1% Nonidet P-40. 1% Triton X-100 and 2% glycerol) or Laemmli sample buffer (62.5 mM Tris-HCl, pH 6.8, 2% SDS, 25% glycerol, 5% β-mercaptoethanol) with freshly added EDTA-free protease inhibitor cocktail (Roche). Protein lysate was either directly subject to immunoblot assay or Co-IP. For Co-IP, Lysates were incubated with FLAG magnetic beads (Sigma) overnight at 4° C. The beads were washed with 1×IP lysis buffer 5 times and then eluted with 2×SDS sample buffer.

### 9. Fluorescence Microscopy

HeLa cell stably expressing GFP-LC3 were washed with PBS two times and fixed with 4% paraformaldehyde (PFA) in PBS on ice for 20 minutes. After washing with PBS three times, cells were mounted with mounting medium (FluorSave reagent, Calbiochem). Cells were examined under Leica invert confocal microscope (TCS-SP8-MP system). Images were taken with 63× oil immersion objective lens at room temperature and image acquisition was performed by LAS X software.

### 10. EGFR Degradation Assay

HEK293T or A549 cells in 6-well plate were washed with PBS two times and serum-starved overnight in DMEM. EGFR endocytosis was induced by incubation with DMEM medium (with 20 mM HEPES and 0.2% BSA) containing 200 ng/mL of EGF (Invitrogen). Cells were collected at each time point after EGF stimulation and lysed in Laemmli sample buffer (62.5 mM Tris-HCl, pH 6.8, 2% SDS, 25% glycerol, 5% f-mercaptoethanol). 20 μg protein lysate was collected for each time point, analyzed by SDS-PAGE and immunoblotted with anti-EGFR antibody (1:2000, Santa Cruz Biotechnology).

### 11. Computational Design of Stapled Peptides

The 3D structure of the α-helical segment corresponding to residues 191-205 within the Beclin1 coiled coil domain (PDB ID 3Q8T; SEQ ID No.: 17) was used as the initial model for SP1 (SEQ ID No.: 1). Eleven other SPs (i.e. SP2-SP12; SEQ ID No.: 2-12) were designed by substituting the residues at positions 191, 194, 195, 201 and 205. A hydrocarbon staple of 13-carbon length was added in silico to link residue 197 and 204. The N-terminal of each SP was capped with an acetyl group and the C-terminal was capped with a methylamide group. All of the above molecular modeling tasks were conducted using the Sybyl software (version 8.0).

A molecular dynamics (MD) simulation was conducted to derive the binding mode of each designed SP (SEQ ID No.: 1-12) to the monomer chain of Beclin1 coiled coil domain. Force field parameters of the stapled region of each SP were prepared using the Antechamber module in the AMBER software (version 14); while the remaining parts of SPs were assigned with FF03SB force field parameters. The complex of Beclin1 and SP (SEQ ID No.: 1-12) was solvated in a TIP3P water box with a margin of 10 Å at each dimension. The complex structure was first optimized through a stepwise process using the Sander module in AMBER, and then was heated up from 0 K to 300 K in 100 ps. Finally, the complex structure was equilibrated without any restraint for 8 ns under 300 K and 1 atm. Based on the outcomes of MD simulation, the MM-GB/SA method implemented in AMBER was used to compute the binding affinity of each designed SP to Beclin1. A total of 400 snapshots were sampled from the last 4 ns segment on the entire MD trajectory with an interval of 10 ps. The final binding energy of each SP was computed as the average of the results obtained on these 400 snapshots. Vibrational entropy was not considered here. All parameters used in the MM-GB/SA computation were set to their default values.

### 12. Synthesis of Stapled Peptide (SP)

The scrambled peptides and SP candidates deemed promising by computational design were acquired commercially from Shanghai ABBiochem Co., Ltd (Shanghai, P.R. China). Chemical structure and purify of the final synthesized products were characterized by HRMS and HPLC.

### Results

### Parallel Coiled Coil Assembly Revealed by a Beclin1-UVRAG Complex Structure

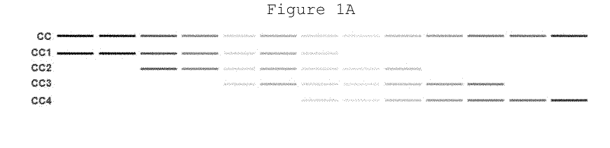

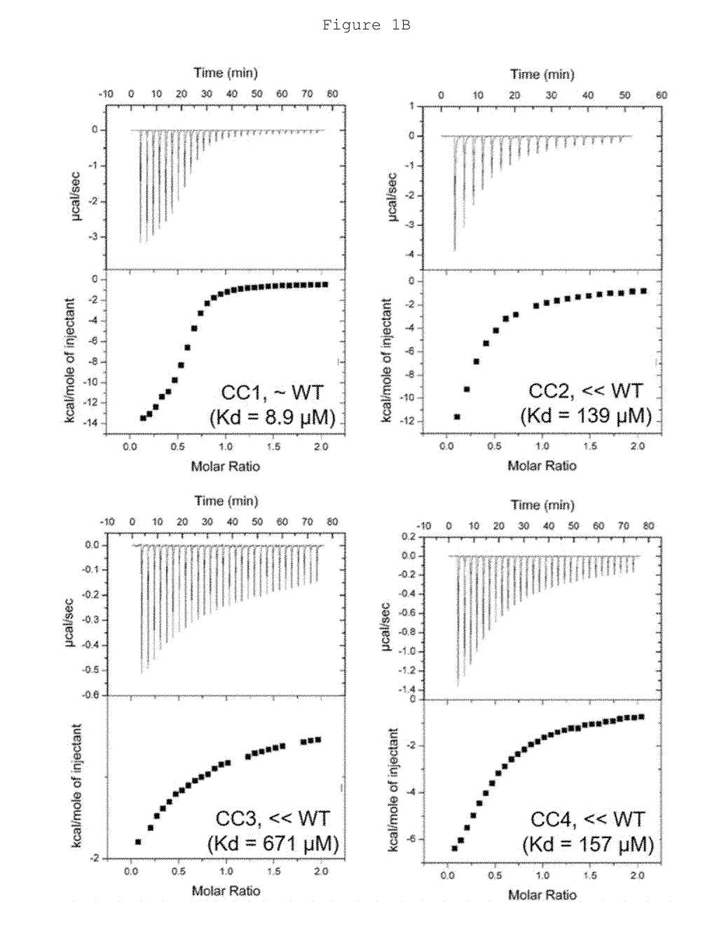

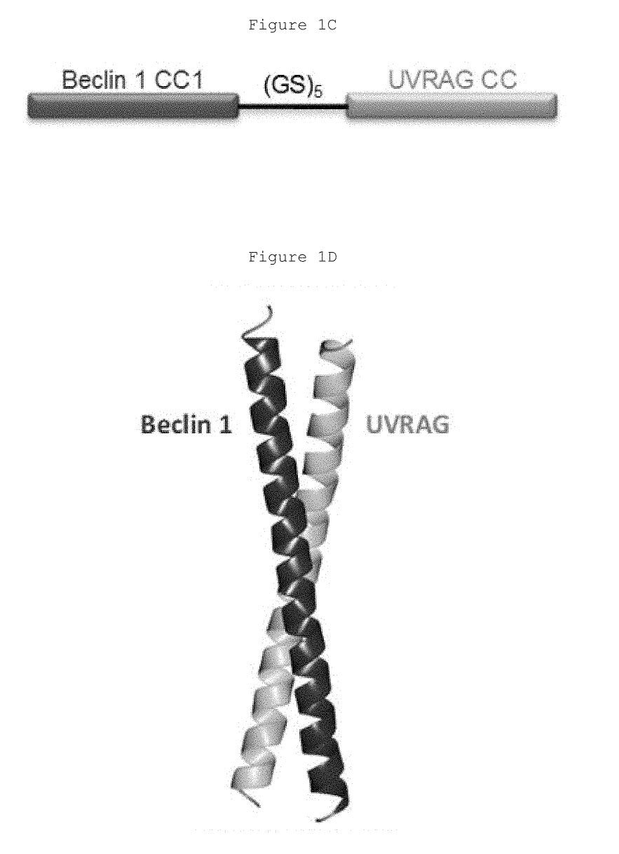

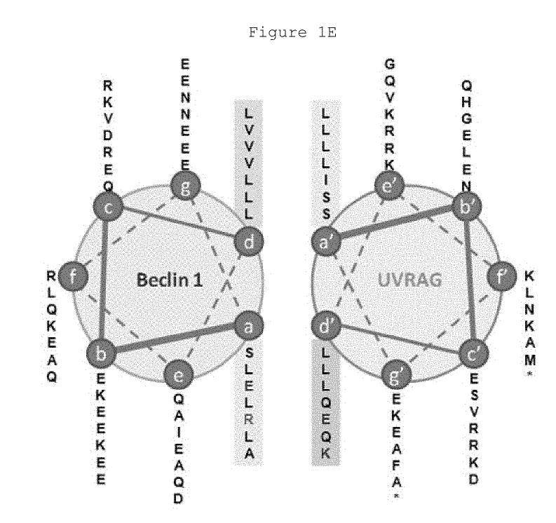

In contrast to the prominent motif of heptad repeat abcdefg displayed by Beclin 1 coiled coil domain over a long stretch of amino acid sequence (˜90 residues), the coiled coil domain of UVRAG shows only short stretches of such repeats (˜50 residues) interspersed by Gly-rich flexible segments. It is not intuitive how Beclin1 and UVRAG would form coiled coil assembly if their sequence motifs don't match well. In order to identify the most critical region within Beclin1 coiled coil domain for UVRAG binding, four Beclin 1 coiled coil constructs (CC1-CC4, SEQ ID No.: 19-22) were generated, each 7 heptad repeats long, that scans through the entire 13 heptad repeats of Beclin1 coiled coil domain (residues 175-266) (FIG. 1A). Isothermal Titration Calorimetry (ITC) measurements reveal that CC1 (SEQ ID No.: 19), i.e. the N-terminal half of the Beclin1 coiled coil domain, interacts strongly with UVRAG with binding affinity Kd comparable to that for the entire Beclin1 coiled coil domain (FIG. 1B). Constructs CC2 to CC4 (SEQ ID No.: 20-22) show significantly weakened interaction with UVRAG, with Kd reduced by 10-50 fold (FIG. 1B). With the most critical region of Beclin1 confirmed, a construct of the Beclin1-UVRAG complex was generated in which the Beclin1 CC1 segment (SEQ ID No.: 19) is tethered to the UVRAG coiled coil domain via a flexible (GS)5 linker (SEQ ID No.: 26) (FIG. 1C). This design is to prevent spontaneous self-assembly among the Beclin 1 and UVRAG coiled coil chains that would interfere with the formation of heterodimeric UVRAG-Beclin 1 complex. This linked construct readily yielded crystals that diffracted to 1.8 Å at synchrotron source and the structure was determined by SIRAS using Au as the heavy metal derivative. (Table 1).

TABLE 2
Sequence number and amino acid sequences of coiled coil domain

| Coiled coil domain | Sequence number | Amino acid sequence |
|---|---|---|
| CC1 | SEQ ID No.: 19 | DSEQAQRELKELALEEERLIQELEDVEKNR |
| | | KVVAENLEKVQAEAERLDQE |
| CC2 | SEQ ID No.: 20 | EERLIQELEDVEKNRKVVAENLEKVQAEAE |
| | | RLDQEEAQYQREYSEFKRQ |
| CC3 | SEQ ID No.: 21 | RKVVAENLEKVQAEAERLDQEEAQYQREY |
| | | SEFKRQQLELDDELKSVENQ |
| CC4 | SEQ ID No.: 22 | AERLDQEEAQYQREYSEFKRQQLELDDELK |
| | | SVENMRYAQMQADKLKKTN |
| CC | SEQ ID No.: 23 | DSEQLQRELKELALEEERLIQELEDVEKNRK |
| | | VVAENLEKVQAEAERLDQEEAQYQREYSE |
| | | FKRQQLELDDELKSVENQMRYAQMQLDKL |
| | | KKTN |
| Coiled coil domain | SEQ ID No.: 24 | TSNELKKESESLRLKILVLRNELERQKKALG |
| of UVRAG | | REVAFLHKQQMALQDKG |
| Coiled coil domain | SEQ ID No.: 25 | MDYKDDDDKKQEEFQKEVLKAMEGKRLT |
| of Atg4L | | DQLRWKIMSCKMRIEQLKQTICKGNEEMK |
| | | KNSEGLLKNKEKNQKLYSRAQRHQEKKEKI |
| | | RHNRKLGDINEKKTIDLKSHYERLARLRR |
| Linked Beclin1 | SEQ ID No.: 26 | DSEQAQRELKELALEEERLIQELEDVEKNR |
| coiled coil fragment | | KVVAENLEKVQAEAERLDQEGSGSGSGSGS |
| (174-223) and | | TSNELKKESESLRLKILVLRNELERQKKALG |
| UVRAG coiled coil | | REVAFLHKQQMALQDKG |
| fragment (228-275) | | |
| with ″(Gly-Ser)5″ | | |
| segment | | |
| Residue 228-298 of | SEQ ID No.: 27 | TSNELKKESESLRLKILVLRNELERQKKALG |
| UVRAG | | REVAFLHKQQMALQDKGSAFSTEHGKLQL |
| | | KDSLSELRKE |

The structure of Beclin1-UVRAG linked construct reveals a parallel heterodimeric coiled coil assembly (FIG. 1D). Within the crystal lattice this complex is formed between two neighboring peptide chains with the Beclin1 CC1 segment (SEQ ID No.: 19) of one molecule matched up against the UVRAG coiled coil region of a symmetry related molecule nearby while the flexible (GS)5 linker is not visible. The Beclin1-UVRAG interface displays the canonical pattern of parallel coiled coil, i.e. residues at a and d positions within the heptad repeat motif form hydrophobic a-a′ and d-d′ pairings to stabilize the heterodimer complex (FIG. 1E). This arrangement is different from the a-d′ pairings observed in the anti-parallel coiled coil homodimer of Beclin1 coiled coil domain alone (Li et al., 2012a).

The Beclin1-UVRAG coiled coil complex was fitted into the crystal structure of the yeast Beclin1-UVRAG complex. The Beclin1 and UVRAG constructs used in the structure of the present invention covers residues 174-223 (SEQ ID No.: 19) and 228-276 (SEQ ID No.: 24), respectively. Based on sequence alignment the corresponding segments in yeast Atg30 and Atg38 are 215-280 and 208-256, matching approximately the first half of their respective CC2 segments and ending right around the area when the Atg30 CC2 strand starts to impinge onto WD40 domain of Vps15. Interestingly, the corresponding CC2 segment of Atg38 shows a small bending around the same area that breaks the α-helical structure of the peptide chain, suggesting that the canonical coiled coil interaction between Atg30 and Atg38 may no longer be sustained beyond the WD40 binding site. Thus the crystal structure manages to capture the most essential segment of the Beclin 1-UVRAG coiled coil assembly.

Beclin1-UVRAG Interface which is Significantly Stabilized by Hydrophobic Pairings and Complementary Interactions

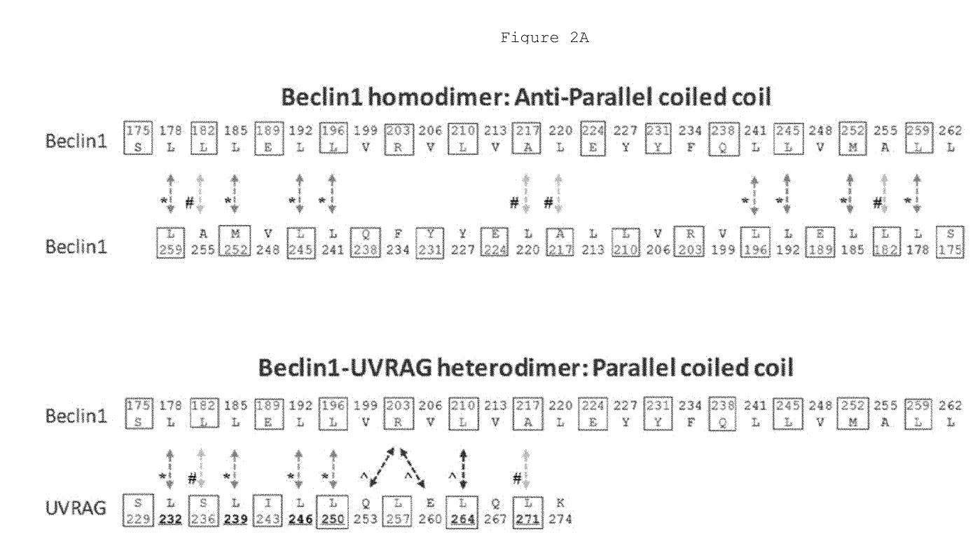

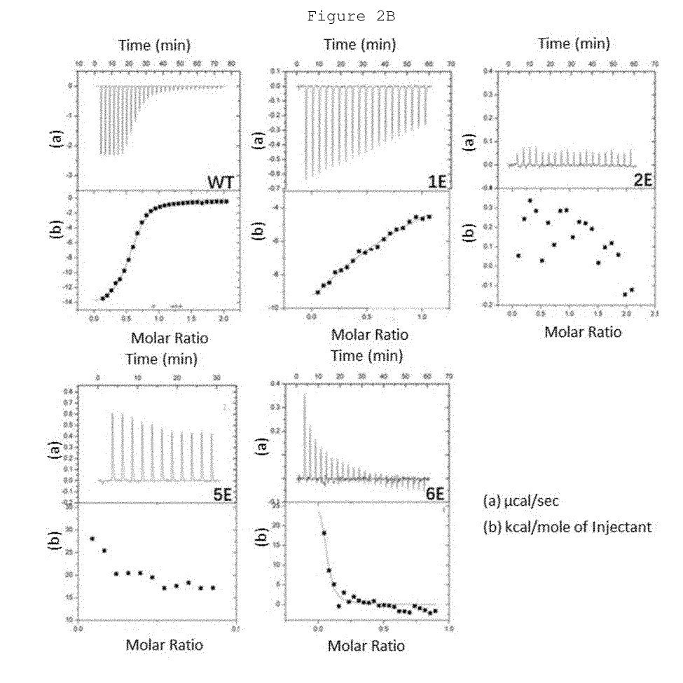

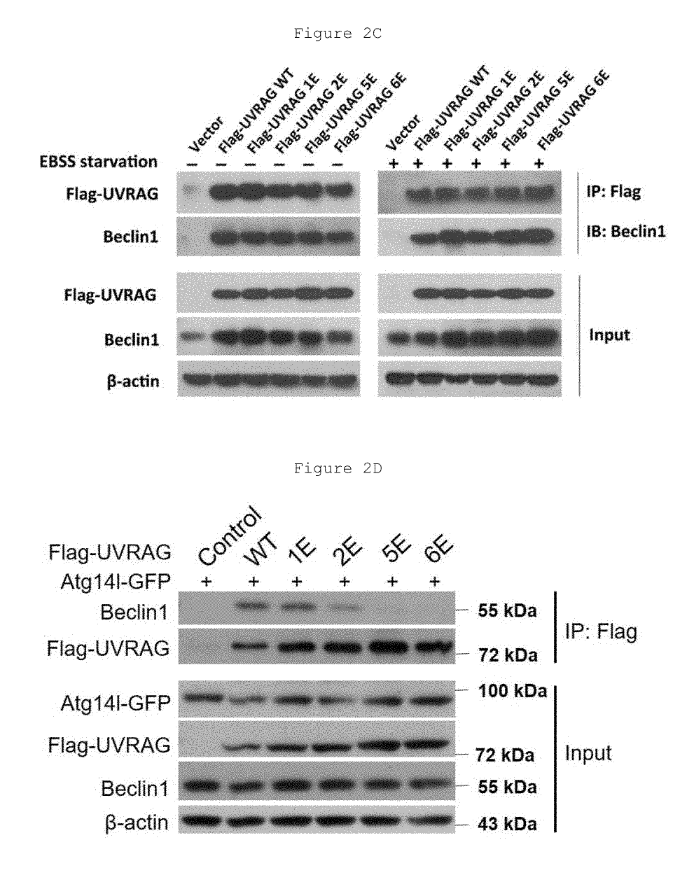

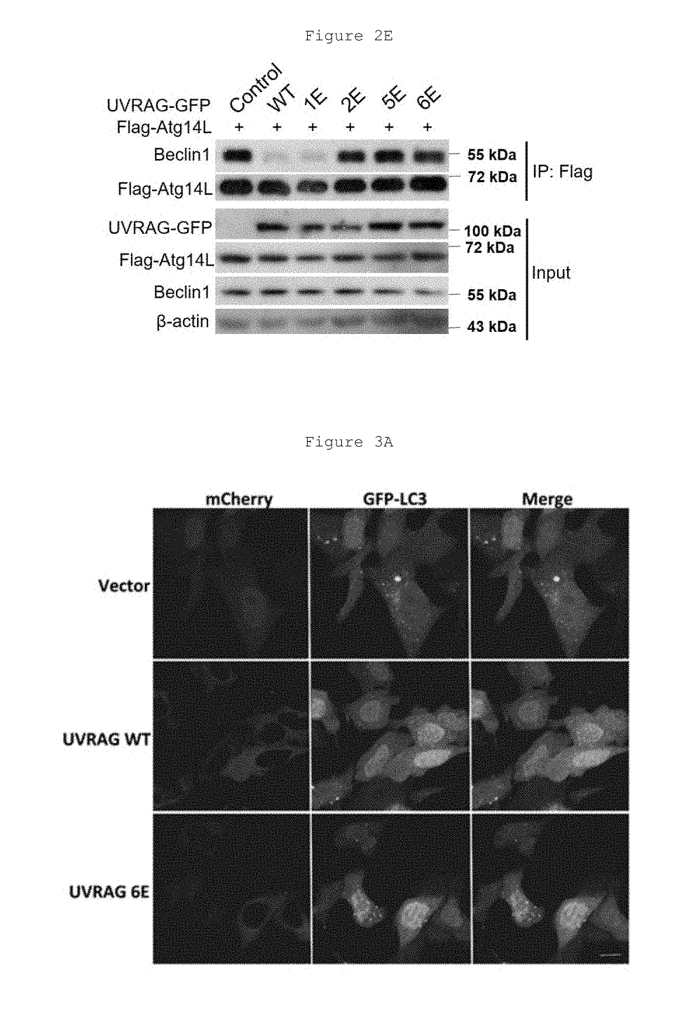

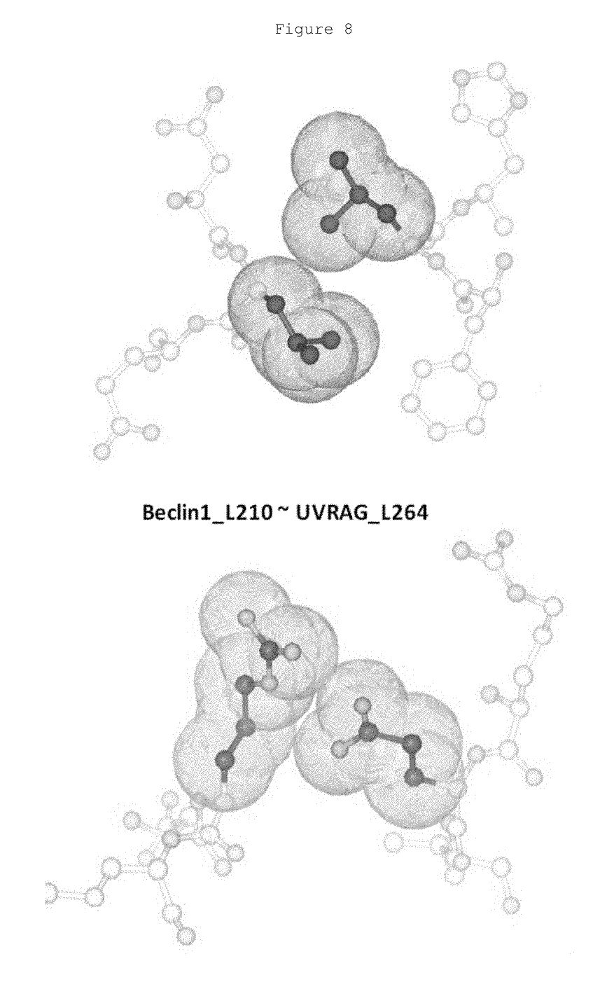

Close analysis of the interface of Beclin1-UVRAG coiled coil complex yields information on the molecular determinants that render the Beclin1-UVRAG heterodimer more stable than the Beclin1 homodimer. First of all, the Beclin1-UVRAG complex contains a series of “perfect” a-a′ and d-d′ parings termed “leucine zippers” at the heterodimer interface to “zip” and stabilize the parallel coiled coil assembly (FIG. 2A). In fact, four such Beclin1-UVRAG “zipper” pairs (L178a-L232a′, L185a-L239a′, L192a-L246a′ and L196d-L250d′, Beclin 1 residues underlined) involve the same Beclin1 residues that form similar hydrophobic a-d′ pairings in Beclin1 homodimer (L178a-L259d′, L185a-M252d′, L192a-L245d′ and L196d-L241a′) (FIG. 2A). Additionally, the Beclin1-UVRAG complex contains several energetically favorable interacting pairs at its coiled coil interface that replace the “imperfect” and destabilizing pairs seen in the Beclin1 homodimer. There is one extra “leucine zipper” pair in Beclin1-UVRAG complex (L210d-L264d′) that replaces the corresponding “imperfect” pair (L210d-Y227d′) in Beclin1 coiled coil homodimer (FIG. 2A and FIG. 8). Furthermore, Beclin1 residue R203 forms electrostatically favorable salt bridge interaction with UVRAG residue E260 at the heterodimer interface (FIG. 2A and FIG. 8). This effectively neutralizes the “destabilizing” effect of R203 exerts on Beclin1 homodimer as found in previous studies (Li et al., 2012a). In summary by retaining all the “perfect” hydrophobic pairings and gaining additional stabilizing interactions the Beclin1-UVRAG complex is notably more stable than the Beclin1 homodimer.

To confirm the structural findings and further delineate the molecular determinants that facilitate stable Beclin1-UVRAG interaction, a series of UVRAG mutants were generated in which one, two, five or all six of leucine residues involved in forming leucine zippers with Beclin1 were replaced by glutamate (Table 2). Isothermal Titration Calorimetry (ITC) results show that the single Leu-to-Glu mutation L246E (termed 1E) already significantly weakens its binding to Beclin1 coiled coil domain in vitro while additional Leu-to-Glu mutations to replace two or more of the leucine residues (termed 2E, 5E and 6E) completely abolish such binding (FIG. 2B). Coimmunoprecipitation (Co-IP) experiments were used to probe the impact of these Leu-to-Glu mutations on Beclin1-UVRAG interaction in vivo. FLAG-tagged UVRAG mutants were transfected into HEK293T cells and their interaction with endogenous Beclin1 was assessed. According to the results obtained, all UVRAG mutants can pull down similar amount of endogenous Beclin1, under both normal and nutrient deprived conditions (FIG. 2C). These results suggest that while UVRAG coiled coil domain is critical for its interaction with Beclin1, weakening or disrupting the Beclin1-UVRAG coiled coil interaction is not sufficient to completely abolish the association of these two proteins in vivo. This may be due to the participation of other regions to facilitate Beclin1-UVRAG association. Indeed, in the crystal structure of the yeast Beclin1-UVRAG complex the N-terminal domains of Atg30 and Atg38 are intertwined at the base of the Y-shape while their C-terminal membrane binding domains are also in close contact at the tip of the regulatory arm (Rostislavleva, Soler et al. 2015).

TABLE 2
Design of UVPAG Leu-to-Glu mutations

| UVRAG | Mutations | Kd |
|---|---|---|
| WT (SEQ ID No.: 28) | None | 0.3 |
| 1E (SEQ ID No.: 29) | L246E | 180 |
| 2E (SEQ ID No.: 30) | L246E_L250E | Not |
| | | detectable |
| 5E (SEQ ID No.: 31) | L232E_L239E_L246E_L250E_L264E | Not |
| | | detectable |
| 6E (SEQ ID No.: 32) | L232E_L239E_L246E_L250E_L264E_L271E | Not |
| | | detectable |

Wild-type UVRAG
(SEQ ID No.: 28)
MSSCASLGGPVPLPPPGPSAALTSGAPARALHVELPSQQRRLRHLRNIAA
RNIVNRNGHQLLDTYFTLHLCDNEKIFKEFYRSEVIKNSLNPTWRSLDFG
IMPDRLDTSVSCFVVKIWGGKEEAFQLLIEWKVYLDGLKYLGQQIHARNQ
NEIIFGLNDGYYGAPCEHGHPNAQKNLLQVDQNCVRNSYDVFSLLRLHRA
QCAIKQTQVTVQRLGKEIEEKLRLTSTSNELKKESECLRLKILVLRNELE
RQKKALGREVAFLHKQQMALQDKGSAFSTEHGKLQLQKDSLSELRKECTA
KRELFLKTNAQLTIRCRQLLSELSYIYPIDLNEHKDYFVCGVKLPNSEDF
QAKEDGSIAVALGYTAHLVSMISFFLQVPLRYPIIHKGSRSITIKDNIND
KLTEKEREFPLYPKGGEKLQFDYGVYLLNKNIAQLRYQHGLGTPDLRQTL
PNLKNFMEHGLMVRCDRHHISNAIPVPKRQSSTFGGADGGFSAGIPSPDK
VHRKRASSENERLQYKTPPPSYNSALTQPGVAMPTSGDSERKVAPLSSSL
DTSLDFSKENKKAGVDLGSSVSGDHGNSDSGQEQGEALPGHLAAVNGTAL
PSEQAGPAGTLLPGSCHPAPSAELCCAVEQAEEIIGLEATGFTSGDQLEA
LSCIPVDSAVAVECDEQVLGEFEEFSRRIYALSENVSSFRRPRRSSDK
UVRAG mutant 1E
(SEQ ID No.: 29)
MSSCASLGGPVPLPPPGPSAALTSGAPARALHVELPSQQRRLRHLRNIAA
RNIVNRNGHQLLDTYFTLHLCDNEKIFKEFYRSEVIKNSLNPTWRSLDFG
IMPDRLDTSVSCFVVKIWGGKEEAFQLLIEWKVYLDGLKYLGQQIHARNQ
NEIIFGLNDGYYGAPCEHKGHPNAQKNLLQVDQNCVRNSYDVFSLLRLHR
AQCAIKQTQVTVQRLGKEIEEKLRLTSTSNELKKESECLRLKILVERNEL
ERQKKALGREVAFLHKQQMALQDKGSAFSTEHGKLQLQKDSLSELRKECT
AKRELFLKTNAQLTIRCRQLLSELSYIYPIDLNEHKDYFVCGVKLPNSED
FQAKEDGSIAVALGYTAHLVSMISFFLQVPLRYPIIHKGSRSTIKDNIND
KLTEKEREFPLYPKGGEKLQFDYGVYLLNKNIAQLRYQHGLGTPDLRQTL
PNLKNFMEHGLMVRCDRHHISNAIPVPKRQSSTFGGADGGFSAGIPSPDK
VHRKRASSENERLQYKTPPPSYNSALTQPGVAMPTSGDSERKVAPLSSSL
DTSLDFSKENKKAGVDLGSSVSGDHGNSDSGQEQGEALPGHLAAVNGTAL
PSEQAGPAGTLLPGSCHPAPSAELCCAVEQAEEIIGLEATGFTSGDQLEA
LSCIPVDSAVAVECDEQVLGEFEEFSRRIYALSENVSSFRRPRRSSDK
UVRAG mutant 2E
(SEQ ID No.: 30)
MSSCASLGGPVPLPPPGPSAALTSGAPARALHVELPSQQRRLRHLRNIAA
RNIVNRNGHQLLDTYFTLHLCDNEKIFKEFYRSEVIKNSLNPTWRSLDFG
IMPDRLDTSVSCFVVKIWGGKEEAFQLLIEWKVYLDGLKYLGQQIHARNQ
NEIIFGLNDGYYGAPCEHKGHPNAQKNLLQVDQNCVRNSYDVFSLLRLHR
AQCAIKQTQVTVQRLGKEIEEKLRLTSTSNELKKESECLRLKILVERNEE
ERQKKALGREVAFLHKQQMALQDKGSAFSTEHGKLQLQKDSLSELRKECT
AKRELFLKTNAQLTIRCRQLLSELSYIYPIDLNEHKDYFVCGVKLPNSED
FQAKEDGSIAVALGYTAHLVSMISFFLQVPLRYPIIHKGSRSTIKDNIND
KLTEKEREFPLYPKGGEKLQFDYGVYLLNKNIAQLRYQHGLGTPDLRQTL
PNLKNFMEHGLMVRCDHHISNAIPVPKRQSSTFGGADGGFSAGIPSPDKV
HRKRASSENERLQYKTPPPSYNSALTQPGVAMPTSGDSERKVAPLSSSLD
TSLDFSKENKKAGVDLGSSVSGDHGNSDSGQEQGEALPGHLAAVNGTALP
SEQAGPAGTLLPGSCHPAPSAELCCAVEQAEEIIGLEATGFTSGDQLEAL
SCIPVDSAVAVECDEQVLGEFEEFSRRIYALSENVSSFRRPRRSSDK
UVRAG mutant 5E
(SEQ ID No.: 31)
MSSCASLGGPVPLPPPGPSAALTSGAPARALHVELPSQQRRLRHLRNIAA
RNIVNRNGHQLLDTYFTLHLCDNEKIFKFPYRSEVIKNSLNPTWRSLDFG
IMPDRLDTSVSCFVVKIWGGKEEAFQLLIEWKVYLDGLKYLGQQIHARNQ
NEIIFGLNDGYYGAPCEHKGHPNAQKNLLOVDQNCVRNSYDVFSLLRLHR
AQCAIKQTQVTVQRLGKEIEEKLRLTSTSNEEKKESECERLKILVERNEE
ERQKKALGREVAFEHKQQMALQDKGSAFSTEHGKLQLQKDSLSELRKECT
AKRELFLKTNAQLTIRCRQLLSELSYIYPIDLNEHKDYFVCGVKLPNSED
FQAKEDGSIAVALGYTAHLVSMISFFLQVPLRYPIIHKGSRSTIKDNIND
KLTEKEREFPLYPKGGEKLQFDYGVYLLNKNIAQLRYQLIGLGTPDLRQT
LPNLKNFMEHGLMVRCDRHHISNAIPVPKRQSSTFGGADGGFSAGIPSPD
KVHRKRASSENERLQYKTPPPSYNSALTQPGVAMPTSGDSERKVAPLSSS
LDTSLDFSKENKKAGVDLGSSVSGDHGNSDSGQEQGEALPGHLAAVNGTA
LPSEQAGPAGTLLPGSLHPAPSAELCCAVECQEEIIGLEATGFTSGDQLE
ALSCIPVDSAVAVECDEQVLGEFEEFSRRIYALSENVSSFRRPRRSSDK
UVRAG mutant 6E
(SEQ ID No.: 32)
MSSCASLGGPVPLPPPGPSAALTSGAPARALHVELPSQQRRLRHLRNIAA
RNIVNRNGHQLLDTYFTLHLCDNEKIFKEFYRSEVIKNSLNPTWRSLDFG
IMPDRLDTSVSCFVVKIWGGKEEAFQLLIEWKVYLDGLKYLGQQIHARNQ
NEIIFGLNDGYYGAPCEHKGHPNAQKNLLQVDQNCVRNSYDVFSLLRLHR
AQCAIKQTQVTVQRLGKEIEEKLRLTSTSNEEKKESECERLKILVERNEE
ERQKKALGREVAFEHKQQMAEQDKGSAFSTEHGKLQLQKDSLSELRKECT
AKRELFLKTNAQLTIRCRQLLSELSYIYPIDLNEHKDYFVCGVKLPNSED
FQAKEDGSIAVALGYTAHLVSMISFFLQVPLRYPIIHKGSRSTIKDNIND
KLTEKEREFPLYPKGGEKLQFDYGVYLLNKNIAQLRYQHGLGTPDLRQTL
PNLKNFMEHGLMVRCDRHHISNAIPVPKRQSSTFGGADGGFSAGIPSPDK
VHRKRASSENERLQYKTPPPSYNSALTQPGVAMPTSGDSERKVAPLSSSL
DTSLDFSKENKKAGVDLGSSVSGDHGNSDSGQEQGEALPGHLAAVNGTAL
PSEQAGPAGTLLPGSCHPAPSAELCCAVEQAEEIIGLEATGFTSGDQLEA
LSCIPVDSAVAVECDEQVLGEFEEFSRRIYALSENVSSFRRPRRSSDK
Monomeric form of Beclin1 coiled coil domain with
L182A mutation
(SEQ ID No.: 33)
DSEQLQREAKELALEEERLIQELEDVEKNRKVVAENLEKVQAEAERLDQE
EAQYQREYSEFKRQQLELDDELKSVENQMRYAQMQLDKLKKTN

To further investigate the impact of these Leu-to-Glu mutations on the potency of the Beclin1-UVRAG interaction, these UVRAG mutants were co-transfected together with Atg14L into HEK293T cells and probed their respective interaction with endogenous Beclin1 by Co-IP experiments. This setup is intended to compare the binding affinity of Atg14L with that of UVRAG mutants as they are competitive and mutually exclusive binding partners of Beclin1. According to the Co-IP results obtained, in the presence of transiently over-expressed Atg14L, both 1E and 2E UVRAG constructs can pull down similar amount of endogenous Beclin1 as compared to wild-type UVRAG (FIG. 2D). However, the 5E and 6E constructs did not pull down detectable amount of Beclin 1, suggesting that the Beclin1-binding potency of these mutants have been weakened and thus cannot compete with Atg14L (FIG. 2D). Conversely. Atg14L cannot pull down Beclin1 in presence of over-expressed wild-type UVRAG or 1E construct (FIG. 2E). However, Atg14L managed to pull down large amount of endogenous Beclin1 when co-expressed with 2E, 5E and 6E constructs (FIG. 2E), indicating that these UVRAG mutants have weakened interaction with Beclin1 and thus cannot compete with Atg14L. In summary, these competitive Co-IP experiments confirm that mutational perturbation of the key hydrophobic residues identified from the Beclin1-UVRAG complex structure leads to significantly weakened interaction between these two molecules with the magnitude of such weakening correlated with the number of mutations incorporated.

Beclin1-UVRAG Interaction Via Coiled Coil Domains which is Required to Promote UVRAG-Dependent Endosome-Lysosomal Degradation of EGFR but not Critical for Autophagy

After the structural and biochemical studies confirm a highly stable Beclin1-UVRAG coiled coil complex underpinned by both hydrophobic and electrostatically favorable pairings at the heterodimer interface, the functional significance of this potent interaction on Vps34-dependent autophagy and endosomal trafficking is studied. This investigation is particularly relevant considering that the Beclin1 coiled coil domain forms only metastable homodimer due to a series of “imperfect” pairings at its otherwise hydrophobic interface (Li, He et al. 2012). It is intriguing whether the potent Beclin1-UVRAG interaction, i.e. a very stable Beclin1-UVRAG coiled coil interface, is required for activities mediated by the UVRAG-containing Beclin1-Vps34 complex.

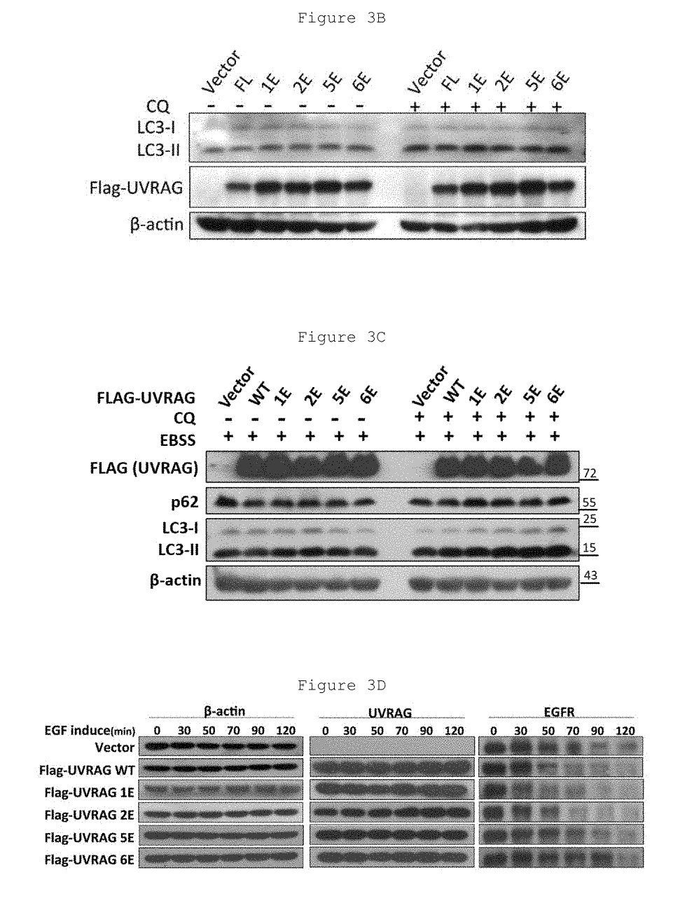

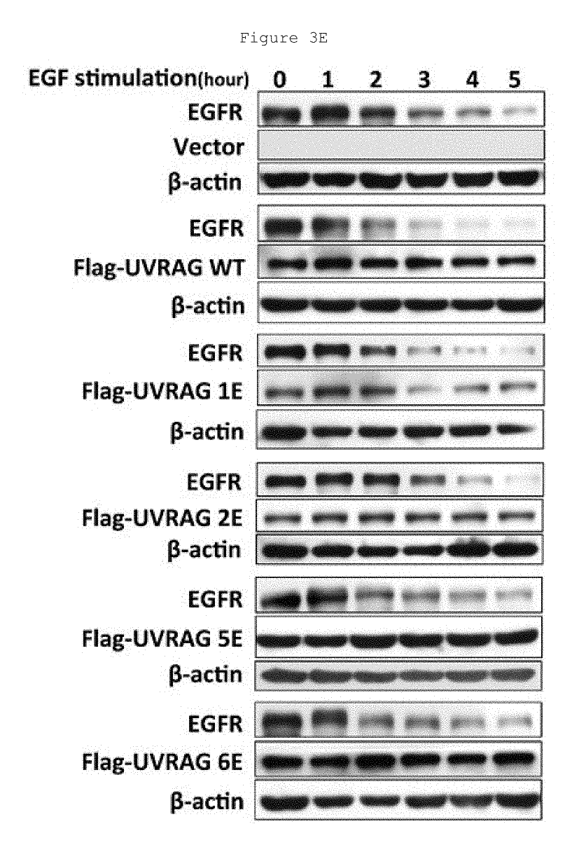

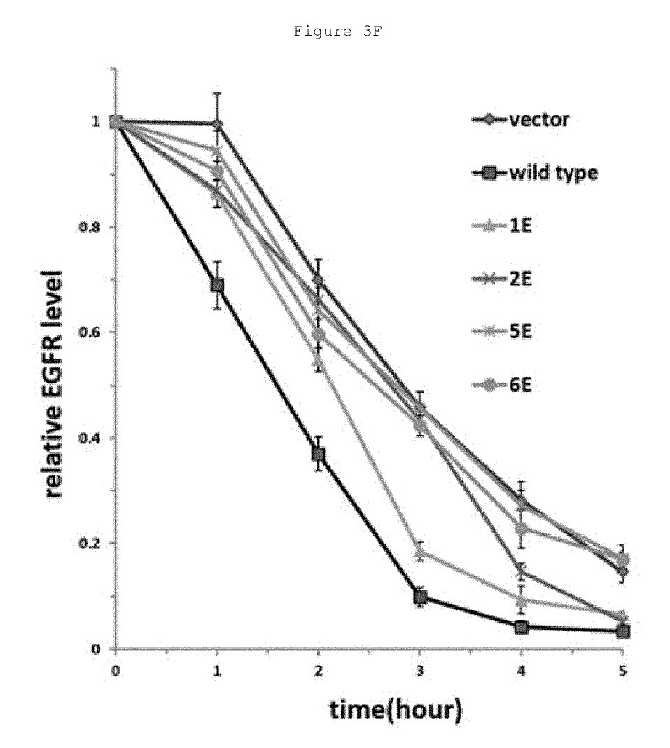

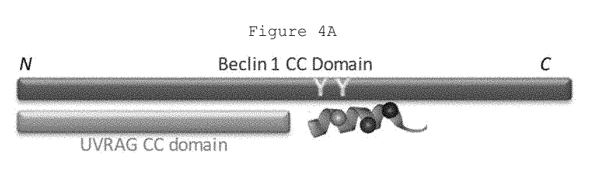

UVRAG mutants (1E to 6E) were transfected into HeLa cells stably expressing GFP-tagged autophagy marker LC3 (GFP-LC3) to assess the impact of Beclin1-UVRAG interaction on autophagy activity. These results show that over-expression of wild-type UVRAG, as well as its mutants, caused no detectable difference in terms of LC3 puncta formation (FIG. 3A). Furthermore, the impact of these UVRAG constructs on autophagic flux in GFP-LC3 is also negligible, as the level of LC3-11 is little changed by wild-type UVRAG or its mutants, whether in the presence or absence of lysosomal inhibitor chloroquine (CQ) (FIG. 3B). Similar results were observed in HEK293T cells, when over-expression of either wild-type UVRAG or UVRAG mutant caused no change in the total amount of p62 or amount of LC3-II (FIG. 3C). These results suggest that neither wild-type UVRAG nor the mutants with weakened Beclin1-UVRAG interaction exert any dominant effect on autophagy process. This finding is in agreement with a previous study by Liang et. al. when the positive effect of UVRAG over-expression in promoting autophagy was only prominent in human HCT116 colon cancer cells that have significantly lower level of endogenous UVRAG due to a truncation mutation, but not in HEK293T or MCF7 cells with normal amount of endogenous UVRAG (McKnight, Zhong et al. 2014).

In addition to its critical role in promoting autophagy induction, UVRAG has been shown to play critical roles in endocytic trafficking, possibly via its interaction with Class C Vps complex as well as being a subunit of the Beclin 1-Vps34 in Beclin 1-UVRAG Complex. To assess the importance of the Beclin1-UVRAG interaction in facilitating endocytic trafficking, the process of epidermal growth factor (EGF)-stimulated endocytic transport and lysosomal degradation of EGF receptor (EGFR) were examined. FLAG-tagged UVRAG constructs were transfected into HEK293T cells and the EGFR degradation process was tracked by immunoblotting. Over-expression of wild-type, 1E or 2E constructs of UVRAG led to significantly enhanced EGFR degradation while 5E or 6E failed to show similar effects (FIG. 3D). To further confirm these findings, similar experiments were conducted using A549 non-small cell lung cancer (NSCLC) cells.

The rate of EGFR degradation in these cells is significantly prolonged as compared to that in HEK293T (half-life ˜3 hours vs. ˜1 hour), probably to sustain excessive proliferation. Nonetheless, over-expression of wild-type UVRAG and 1E construct in A549 significantly enhanced the degradation profile of EGFR, shortening the half-life to ˜2 hours with less than 10% remaining after 5 hours (FIG. 3E). 2E construct showed a weaker effect, with half-life comparable to that for control, but the overall degradation after 5 hours was improved (˜5% remaining vs. 20% for control). However, 5E and 6E constructs did not show any promotional effect. Their EGFR degradation profiles are largely identical to that for control (FIG. 3E). These data suggest that the promotional effect of UVRAG on EGFR degradation is modulated by the potency of the Beclin 1-UVRAG interaction. Only strong interactions afforded by wild-type or 1E construct led to significant enhancement to EGFR degradation. Weaker constructs like 2E only induced subdued effects while constructs like 5E and 6E that are severely weakened cannot induce any promotion.

### Structure-Based Rational Design of Beclin1-Targeting Stapled Peptides

Given the importance of the Beclin1-UVRAG interaction in facilitating lysosomal degradation of EGFR, small-molecule compounds were designed in the present invention to target the Beclin 1 coiled-coil domain and promote EGFR degradation. Such compounds would have the translational potential to be developed into a novel approach to suppress EGFR-driven proliferation, for example, in cancer cells.

Considering that the Beclin1 coiled coil domain is essentially a long α-helix and lacks distinct structural features to constitute a conventional binding site for typical small-molecule compounds, hydrocarbon stapled peptide can be used as the scaffold for the designed molecules. This type of peptides mimetics contains hydrocarbon links that “staple” residues together to stabilize their α-helical structure and have been proven as an effective approach to modulate protein-protein interactions. Besides, hydrocarbon stapled peptides are generally more cell-permeable and thus more “drug-like”.

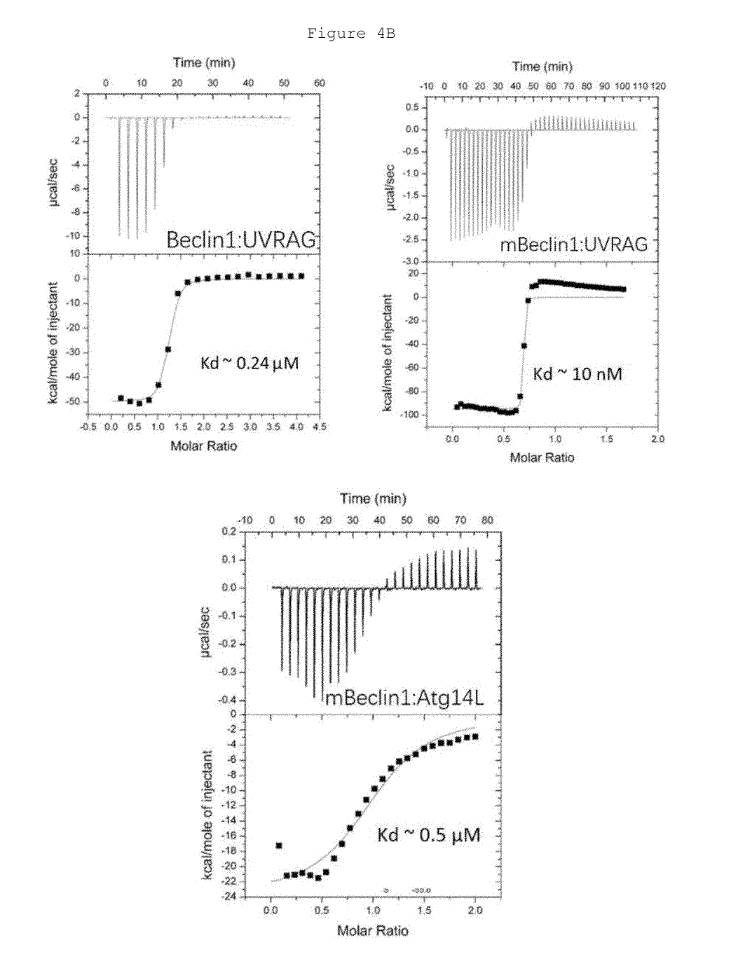

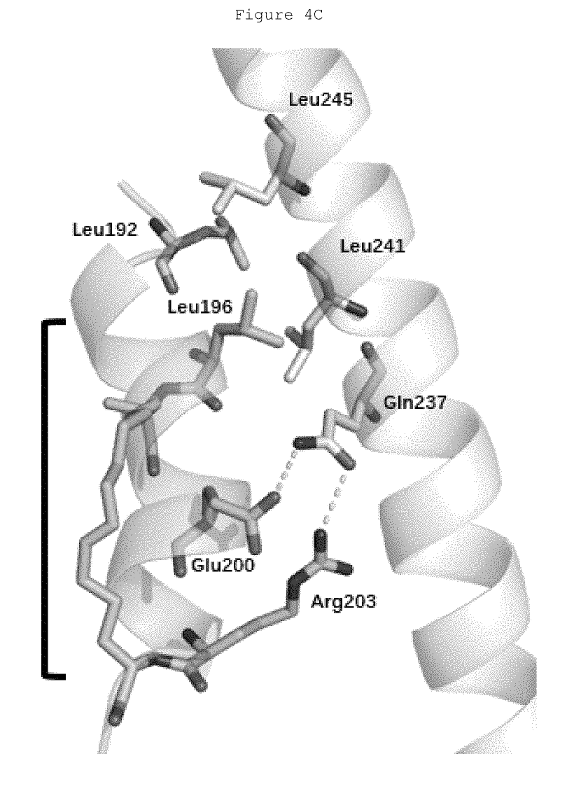

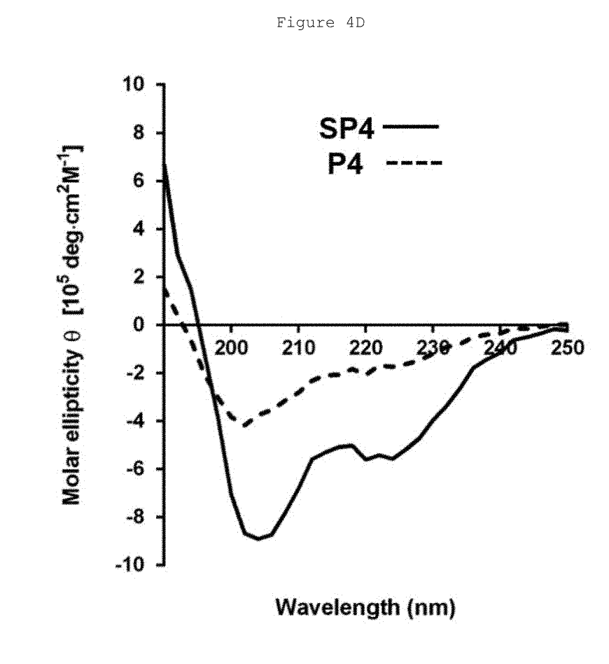

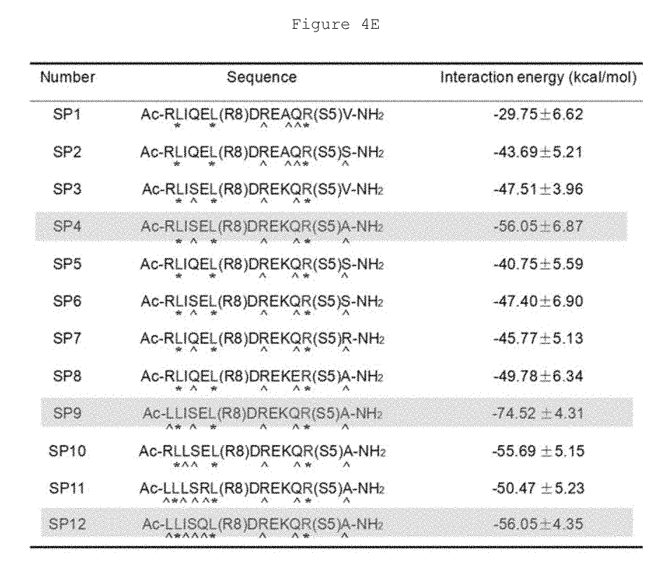

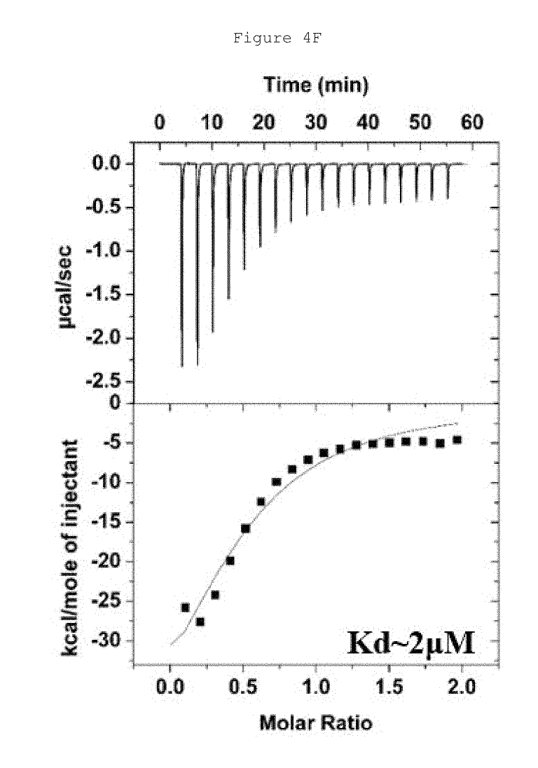

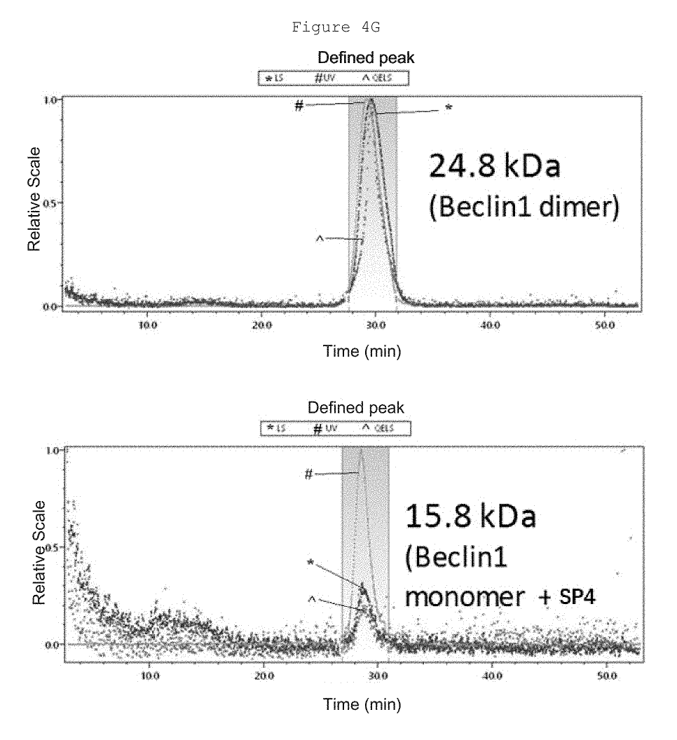

In terms of binding site for these stapled peptides, it is desirable to target them specifically to the C-terminal portion of Beclin1 coiled-coil domain because this region forms part of the Beclin1 homodimer interface but is not involved with UVRAG interaction (FIG. 4A). Stapled peptides binding to this site are expected to disrupt the metastable dimerization of Beclin1 coiled coil domain and render Beclin1 monomeric. This may lead to more effective Beclin1-Atg14L/UVRAG interaction to promote autophagy and enhance lysosomal degradation of EGFR.

In particular, the binding site were narrowed down for stapled peptides to the region of residues 231-245 (SEQ ID No.: 15) based on the previous findings and the Beclin1-UVRAG structure. The starting point was set at Y231 as this residue, together with nearby Y235, corresponds to the two tyrosine residues phosphorylated by EGFR in human Beclin1 to blunt lysosomal degradation of EGFR and sustain tumorigenesis. The next residue S232 is also a phosphorylation site targeted by Akt that functions to inhibit autophagy and facilitates Akt-driven tumorigenesis. A stapled peptide binding to this region may interfere with these phosphorylation events and reduce their negative impact on autophagy. The ending point for the binding site was set at L245 because this residue, and L241 nearby, have been shown by the previous study to form hydrophobic leucine zipper pairs with L192 and L196 at the N-terminal part of Beclin1 coiled coil domain to promote its homodimerization. Furthermore, a Beclin1 mutant L241E/L245E were generated to weaken these leucine zipper pairs binds to UVRAG (SEQ ID No.: 24) with Kd of ˜10 nM, about 20 times stronger as compared to the Kd of ˜0.24 μM for wild-type (FIG. 4B), suggesting that blocking these C-terminal leucine residues with stapled peptides would be beneficial for the Beclin1-UVRAG interaction.

With the target binding site of residues 231-245 (SEQ ID No.: 15) defined, the design of a small library of stapled peptides were proceeded. The model of the first stapled peptide (SP1, SEQ ID No.: 1) was built by simply taking the α-helical segment that interacts with the target region within the Beclin1 homodimer structure, i.e. the segment covering residues 191-205 (SEQ ID No.: 17), as the prototype. A hydrocarbon staple was introduced in silico to link residues 197 and 204, both located on the “outer” side of the helix and not involved in coiled coil interface, to help stabilize the α-helical structure but not to interfere with Beclin1 binding. The structural model of SP1 (SEQ ID No.: 1) binding to Beclin1 was generated simply by superposing SP1 (SEQ ID No.: 1) onto the Beclin1 coiled coil homodimer structure (FIG. 4C). Computational optimization to enhance the binding affinity of SP1 (SEQ ID No.: 1) toward the target region was carried out. A library of stapled peptides (SP2-SP12; SEQ ID No.: 2-12) was generated in which residues deemed critical for target site binding were unchanged while other amino acid residues were computationally varied (FIG. 4E). The binding modes of these stapled peptides to the Beclin1 molecule were characterized by molecular dynamics (MD) simulations and their binding energies were computed using the force field-based MM-GB/SA method. Certain sequence changes, such as replacing Gln194 with Ser and Val205 with Ala in SP4 (SEQ ID No.: 4), led to significantly improved binding energy (FIG. 4E).

Tat sequence (SEQ ID No.: 13: YGRKKRRQRRR) was linked in front of all peptides except the rhodamine B labeled one to enhance cell permeability. The computationally optimized stapled peptide SP4 (SEQ ID No.: 4) was chosen and synthesized by a commercial vendor following the synthetic method pioneered by Kim et. al. (Kim, Grossmann et al. 2011) (FIG. 4E). The purified product was confirmed by mass spectroscopy and HPLC. The importance of the hydrocarbon staple in maintaining the α-helical structure of the designed peptide was confirmed by circular dichroism (CD) measurements (FIG. 4F). The CD spectrum of peptide P4, which is the same as SP4 (SEQ ID No.: 4) but without the hydrocarbon staple, showed largely loop-like profile. The CD spectrum of SP4 (SEQ ID No.: 4), however, revealed high α-helical content. ITC profile showed direct interaction between SP4 (SEQ ID No.: 4) and Beclin1 coiled coil domain with Kd˜2 μM, suggesting that this molecule can bind to Beclin1 coiled coil domain effectively and most likely at the intended target region (FIG. 4F). Furthermore, SP4 (SEQ ID No.: 4) can induce dimer-to-monomer transition in Beclin1 coiled coil domain. The Light Scattering (LS) profile of Beclin1 coiled coil domain in absence of SP4 (SEQ ID No.: 4) indicates a homodimer with predicted molecular weight of 24.8 kDa. However, the presence of SP4 (SEQ ID No.: 4) caused Beclin1 coiled coil domain to adopt monomeric form as the molecular weight predicted from LS profile became 15.8 kDa (FIG. 4G).

In one embodiment, examples of peptides include, but not limited to, the peptides described in FIG. 4E. In one embodiment, a stapled peptide (1) with a particular amino acid sequence is designed to target the Beclin1 coiled coil region spanning residues 231 to 245 (SEQ ID No.: 15). A two-turn hydrocarbon staple is added to stabilize the α-helical structure of the designed peptide. In some embodiments, mutated analogs of peptide (1) are designed.

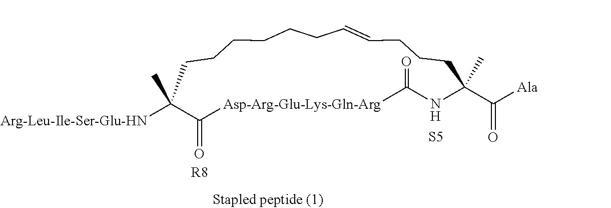

In summary, structure-based design of stapled peptides that mimic the Beclin1 segment of residues 191-205 (SEQ ID No.: 17) can bind to Beclin1 coiled coil domain with high affinity and render it monomeric to promote Beclin1-UVRAG interaction. SEQ ID No.: 17 corresponds to amino acids 193-207 of human Beclin 1 (SEQ ID No.: 18).

### Beclin1-Specific Stapled Peptide Promotes Autophagy and Enhances Lysosomal Degradation of EGFR

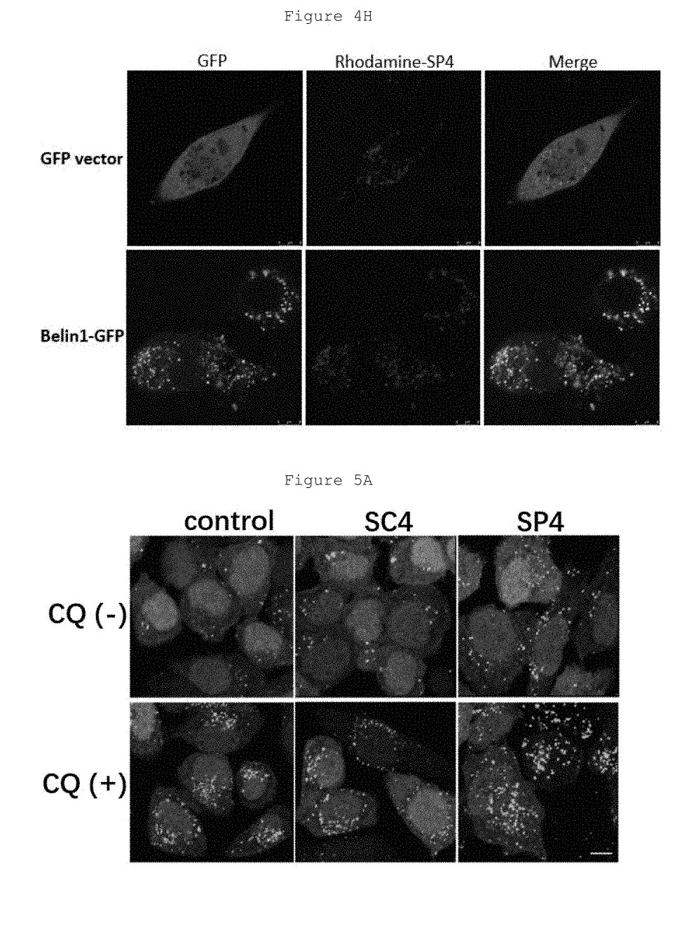

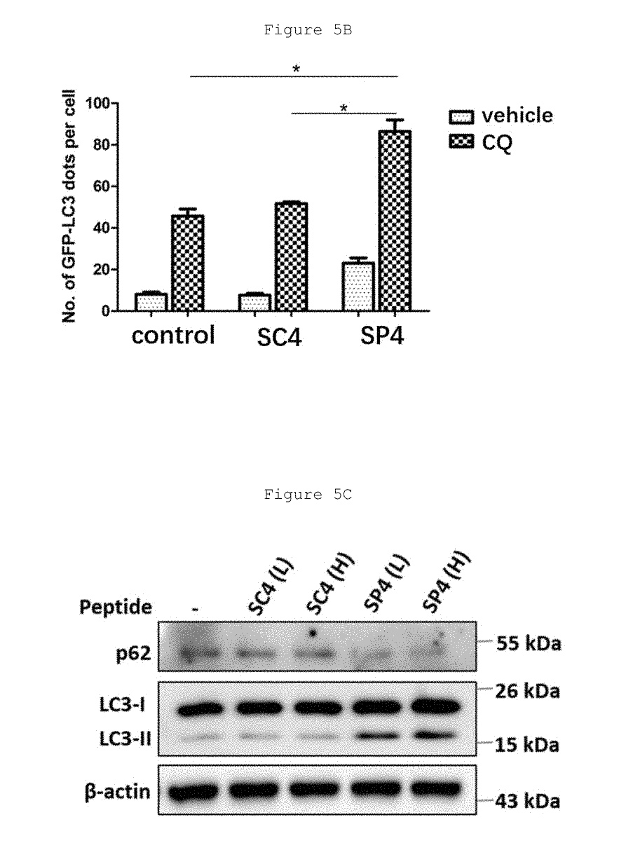

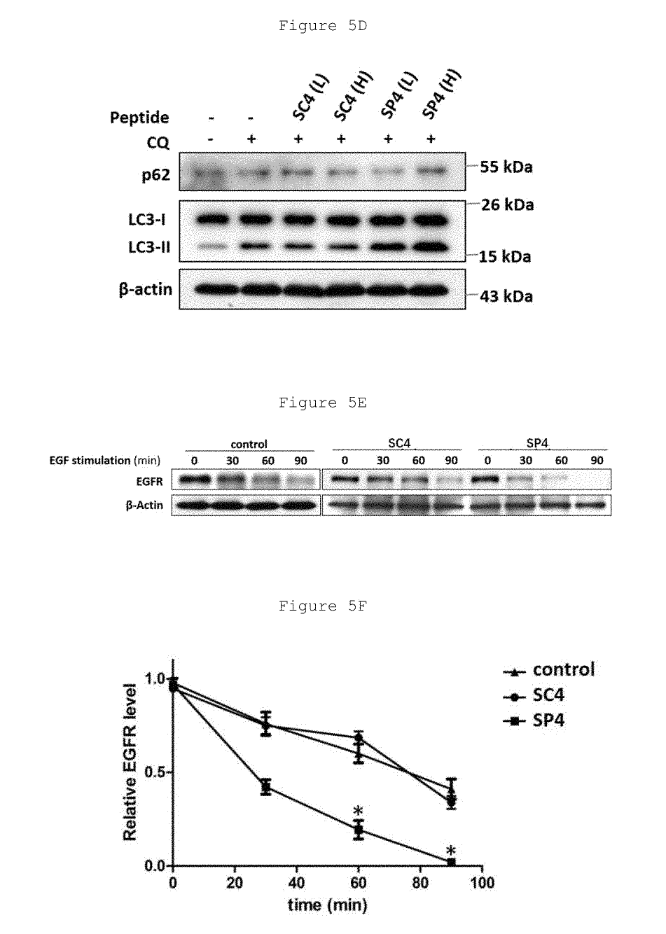

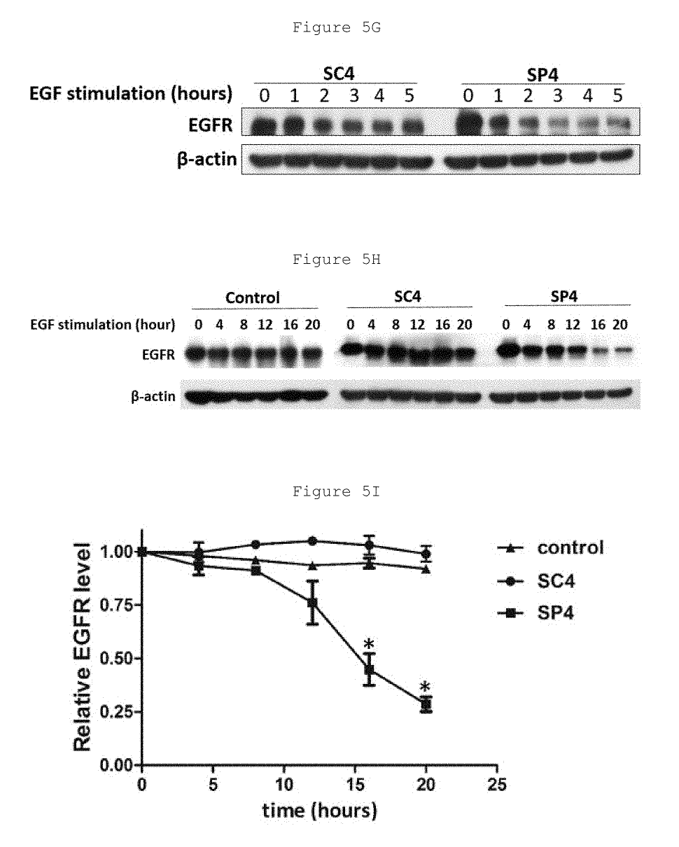

The biological efficacy of the designed peptide SP4 (SEQ ID No.: 4) in modulating autophagy and lysosomal degradation of EGFR was characterized using cell-based assays. To enhance cell permeability, the HIV Tat sequence (SEQ ID No.: 13) were appended to SP4 (SEQ ID No.: 4) (Tat-stapled) and added it to HeLa cells stably expressing GFP-LC3. A Tat-scrambled peptide was used as control for this experiment in which the sequence of SP4 (SEQ ID No.: 4) was scrambled into random order, without hydrocarbon stable, and appended after the Tat sequence. The results of the present invention showed that Tat380 stapled peptide induced significantly larger number of LC3 puncta as compared to both control and Tat-scrambled, both in the presence and absence of chloroquine (FIGS. 5A and 5B). Similarly, Tat-stapled peptide also led to higher LC3 lipidation rate in these HeLa cells, particularly in the presence of lysosomal inhibitor CQ (FIG. 5C).

The efficacy of SP4 (SEQ ID No.: 4) was tested in terms of promoting autophagy in NSCLC cells. Rhodamine-labeled SP4 co-localized well with GFP-Beclin 1 in A549 NSCLC cells (FIG. 4H). Treatment of HEK293T cells with SP4 (SEQ ID No.: 4) led to enhanced LC3 lipidation in dosage-dependent manner in the absence or presence of chloroquine (CQ) (FIGS. 5C and 5D). Furthermore, the efficacy of SP4 (SEQ ID No.: 4) was tested in regulating EGFR degradation. Addition of SP4 (SEQ ID No.: 4) to HEK293T cells significantly enhanced EGFR degradation with half-life shortened from more than 90 minutes in the case of control or scrambled peptide to shorter than 30 minutes for SP4 (SEQ ID No.: 4) (FIGS. 5E and 5F). Moreover. SP4 (SEQ ID No.: 4) treatment significantly enhanced EGFR degradation in NSCLCs bearing wild type EGFR (A549 cell line, FIG. 5G) or mutated EGFR (H1975 cell lines. FIGS. 5H and 51I).

In summary, structure-based rational design targeting the Beclin1 coiled coil domain at region 231-245 (SEQ ID No.: 15) has yielded stapled peptides that specifically bind to Beclin1 coiled coil domain and render it monomeric to promote Beclin1-UVRAG interaction. SEQ ID No.: 15 corresponds to amino acids 233-247 of human Beclin1 (SEQ ID No.).

Collectively, the data of the present invention confirmed that the rationally designed stapled peptide SP4 (SEQ ID No.: 4) can promote autophagy activity and enhance EGFR degradation in a Beclin1-dependent manner.

### DISCUSSION

The direct interaction between Beclin1 and its two mutually competitive binding partners Atg14L and UVRAG is essential for the formation of functionally distinct Atg14L- or UVRAG-containing Beclin1-Vps34 subcomplexes. Interestingly. Beclin1, Atg14L and UVRAG all contain a coiled coil domain that is critical for their respective interactions. It is tempting to propose that these domains can facilitate stable Beclin1-Atg14L/UVRAG interaction by simply “wrapping” around each other to form coiled coil assemblies. But the molecular mechanism of their specific interactions is not known. In particular, the coiled coil domains of all three proteins contain prominent “imperfect” features, i.e. charged or polar residues are frequently found at a and d positions within the heptad repeat motif where hydrophobic residues are expected. As a result, the coiled coil domains of Atg14L and UVRAG are actually monomeric in vitro while the coiled coil domain of Beclin1 only forms a metastable homodimer. It is not intuitive how these “imperfect” coils can form stable Beclin1-Atg14L/UVRAG heterodimeric assemblies.

The present specification presents crystal structure of the Beclin1-UVRAG complex, showing that while the Beclin1-UVRAG heterodimer is highly similar to the Beclin1 homodimer by forming almost identical set of leucine zipper pairs at the coiled coil interface, it does hold a clear advantage in terms of handling “imperfect” residues. Specifically, Beclin residue R203 and UVRAG residue E260, located at a and d positions within their respective heptad repeat motif, are two major “destabilizing” factors because of their charged side chains. However, this pair of residues are brought together in the Beclin1-UVRAG complex and form electrostatically favorable interaction via direct salt bridge to stabilize the heterodimeric coiled coil interface. Thus, “imperfect” residues of Beclin1 and UVRAG coiled coil domains, through sequence complementarity, become the defining features to render the Beclin 1-UVRAG interaction more potent than Beclin1 homodimer. Similar mechanism may also be at play for the Beclin1-Atg14L interaction, i.e. complementarity between “imperfect” Beclin1 and Atg14L residues would favor their heterodimeric coiled coil assembly over the functionally inactive Beclin1 homodimer.

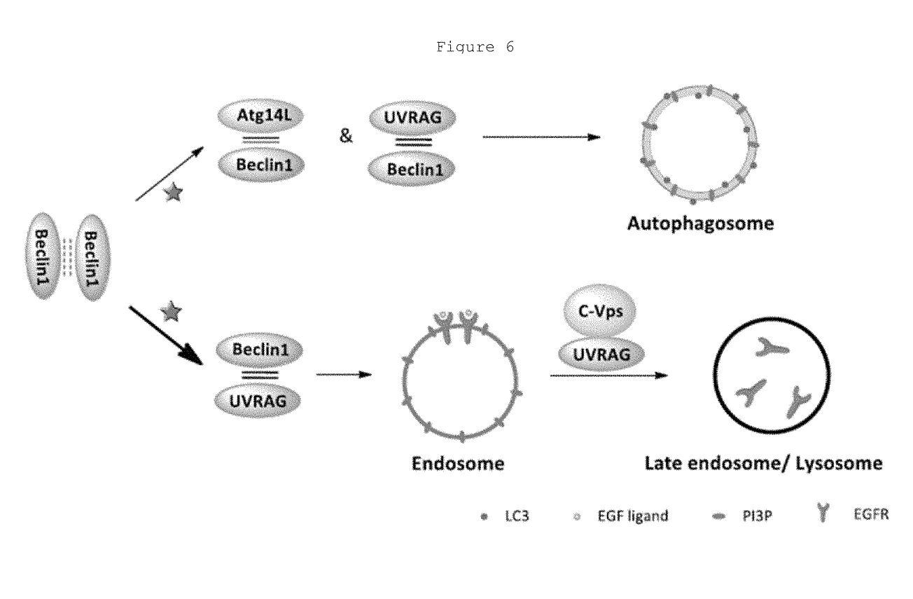

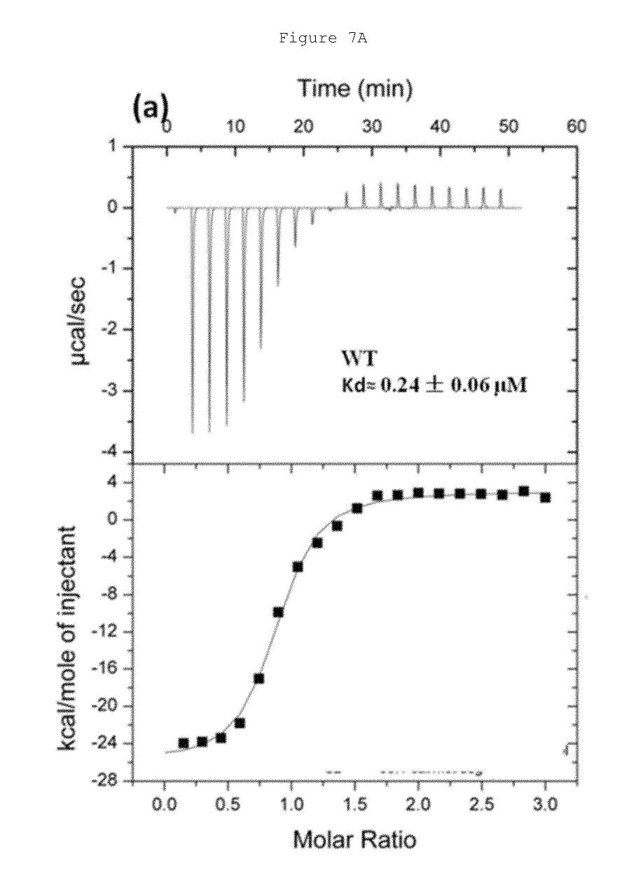

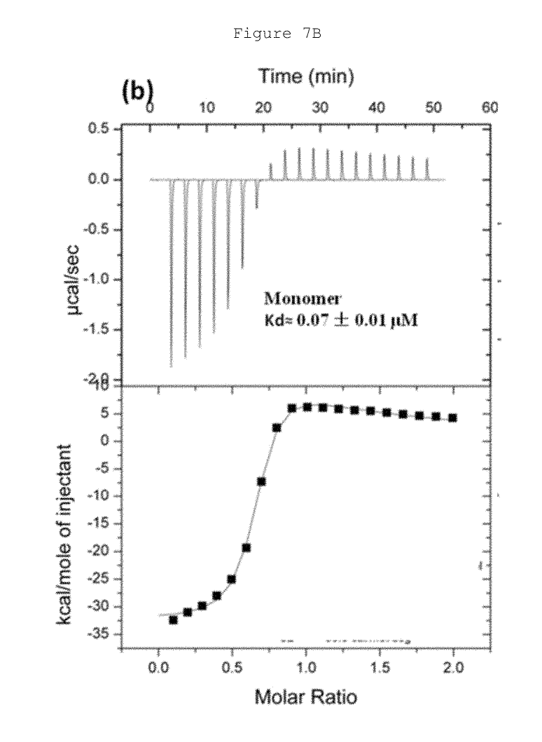

The structure-based functional studies of the present invention also reveal that the potency of the Beclin 1-UVRAG interaction mediated by their respective coiled coil domains is critical for promoting Vps34-dependent endosomal processes. Only UVRAG constructs that have strong binding affinity for Beclin1, i.e. wild-type and 1E, 2E mutants, can effectively promote lysosomal degradation of EGFR when over-expressed. UVRAG mutants like 5E and 6E fail to do so even though they retain association with Beclin in vivo. This requirement on potency is likely due to the competition UVRAG faces from either Atg14L or Beclin1 homodimer in terms of forming UVRAG-containing Beclin1-Vps34 complex. First of all. Atg14L and UVRAG are mutually exclusive binding partners for Beclin 1 via their respective coiled coil domains. Stronger binding affinity by UVRAG is necessary to out-compete Atg14L for the UVRAG-Vps34 complex. Additionally, previous study has proposed that excessive amount of Beclin1 may exist as a reserve pool in functionally inactive homodimeric form. Over-expressed UVRAG with stronger Beclin1 binding affinity may disrupt the metastable Beclin1 homodimer and form UVRAG-containing Beclin1-Vps34 complexes to promote Vps34-dependent processes like endocytic trafficking (FIG. 6). The results of the present invention show that this scenario is more likely than outright competition with Atg14L because over-expression of UVRAG did not affect Atg14L-dependent autophagy activity, suggesting that the amount of Atg14L-containing Beclin1-Vps34 complex is not affected.

UVRAG is a multivalent effector of the endocytic trafficking process and can regulate lysosomal degradation of EGFR through at least two distinct routes. On one hand, the UVRAG-containing Beclin1-Vps34 complex can lead to increased PI3P production and assist maturation of EGFR-containing endosomes. On the other hand, UVRAG also interacts with Class C Vps complex to promote fusion of autophagosomes or early endosomes with late endosomes/lysosomes to enhance lysosomal degradation of EGFR. These two interactions are genetically separable because UVRAG binds to Beclin1 via its coiled coil domain but uses its N-terminal C2 domain to interact with Class C Vps complex. However, the relationship between these two routes is not clear. In the present invention, all UVRAG mutants are expected to retain their interaction with Class C Vps complex. Hence the distinct phenotypes in terms of regulating lysosomal degradation of EGFR arise solely from their different binding affinities to Beclin 1. The enhancement effect observed in 1E and 2E mutants but not in 5E or 6E suggests that the role of UVRAG in endocytic trafficking mediated via the Beclin1-UVRAG interaction is upstream of that mediated via the Class C Vps-UVRAG interaction and probably dominants over it too (FIG. 6).

Lastly, there is intense interest to target the autophagy process for disease modifying therapies. Multiple clinical trials were initiated using autophagy inhibitor CQ in combination with existing cancer drugs to enhance therapeutic efficacy for late-stage refractory cancer types. However, potent and specific modulators of autophagy are lacking because compounds like CQ and mTOR inhibitors are not specific to autophagy and may have off-target effect. A previous study reported a Beclin1 peptide derived from its membrane-binding region can serve as potent inducer of autophagy and decrease the replication of pathogens in cell- and animal-based models. Here a new strategy is presented for generating Beclin1 peptides for autophagy modulation. By specifically targeting the Beclin1 coiled coil domain C-terminal to the UVRAG binding site, rationally designed Beclin1 peptides with hydrocarbon staples to stabilize their α-helical structure can bind to functionally inactive Beclin homodimer in the reserve pool, assist its dimer-to-monomer transition and promote the formation of Atg14L/UVRAG containing Beclin1-Vps34 complexes (FIG. 6). As a result, both Vps34-dependent autophagy and endocytic trafficking can be enhanced, resulting in enhanced lysosomal degradation of EGFR and possibly inhibition of EGFR-driven cancer cell proliferation.

The approach of the present invention provides a novel Beclin1-specific strategy to target the Beclin1-Vps34 complex for EGFR-based anti-cancer treatment. Furthermore, as recent studies have implicated the UVRAG-containing Beclin1-Vps34 complex in endocytic degradation of multiple membrane receptors such as insulin receptor (IR) and TGF-β receptor ALK5, the design strategy presented herein can be applied to these processes as well.

### REFERENCES

1. (2002). High-throughput structure determination. Proceedings of the 2002 CCP4 (Collaborative Computational Project in Macromolecular Crystallography) study weekend. January, 2002. York, United Kingdom. Acta Crystallogr D Biol Crystallogr 58, 1897-1970.
2. (2002). High-throughput structure determination. Proceedings of the 2002 CCP4 (Collaborative Computational Project in Macromolecular Crystallography) study weekend. January, 2002. York, United Kingdom. Acta Crystallogr D Biol Crystallogr 58, 1897-1970.
3. Adi-Harel, S., Erlich, S., Schmukler, E., Cohen-Kedar, S., Segev, O., Mizrachy, L., Hirsch, J. A., and Pinkas-Kramarski, R (2010). Beclin 1 self-association is independent of autophagy induction by amino acid deprivation and rapamycin treatment. J Cell Biochem 110, 1262-1271.
4. Emsley, P., and Cowtan, K. (2004). Coot: model-building tools for molecular graphics. Acta Crystallogr D Biol Crystallogr 60, 2126-2132.
5. He, C., and Levine, B. (2010). The Beclin 1 interactome. Curr Opin Cell Biol 22, 140-149.
6. Li, X., He, L., Che, K. H., Funderburk, S. F., Pan, L., Pan, N., Zhang, M., Yue, Z., and Zhao, Y (2012a). Imperfect interface of Beclin1 coiled-coil domain regulates homodimer and heterodimer formation with Atg14L and UVRAG. Nat Commun 3, 662.
7. Li, X., He, L., Zhang, M., Yue, Z., and Zhao, Y (2012b). The BECN1 coiled coil domain: an “imperfect” homodimer interface that facilitates ATG14 and UVRAG binding. Autophagy 8, 1258-1260.
8. Liang, C., Feng, P., Ku, B., Dotan, I., Canaani, D., Oh, B. H., and Jung, J. U. (2006). Autophagic and tumour suppressor activity of a novel Beclin 1-binding protein UVRAG. Nat Cell Biol 8, 688-699.
9. Liang, C., Feng, P., Ku, B., Oh, B. H., and Jung. J. U. (2007). UVRAG: a new player in autophagy and tumor cell growth. Autophagy 3, 69-71.
10. Liang, C., Lee, J. S., Inn, K. S., Gack, M. U., Li, Q., Roberts, E. A., Vergne, I., Deretic, V., Feng. P., Akazawa, C., et al. (2008a). Beclin1-binding UVRAG targets the class C Vps complex to coordinate autophagosome maturation and endocytic trafficking. Nat Cell Biol 10, 776-787.
11. Liang, C., Sir, D., Lee, S., Ou, J. H., and Jung, J. U. (2008b). Beyond autophagy: the role of UVRAG in membrane trafficking. Autophagy 4, 817-820.
12. Matsunaga, K., Saitoh, T, Tabata, K., Omori, H., Satoh, T., Kurotori, N., Maejima, I., Shirahama-Noda, K., Ichimura, T., Isobe, T., et al. (2009). Two Beclin 1-binding proteins, Atg14L and Rubicon, reciprocally regulate autophagy at different stages. Nat Cell Biol 11, 385-396.
13. Murshudov, G. N., Vagin, A. A., and Dodson, E. J. (1997). Refinement of macromolecular structures by the maximum-likelihood method. Acta Crystallogr D Biol Crystallogr 53, 240-255.
14. Noble, C. G., Dong. J. M., Manser, E., and Song, H. (2008). Bcl-xL and UVRAG cause a monomer-dimer switch in Beclin 1. J Biol Chem 283, 26274-26282.
15. Perelman, B., Dafni, N., Naiman. T., Eli, D., Yaakov. M., Feng, T. L., Sinha, S., Weber, G., Khodaei, S., Sancar, A., et al. (1997). Molecular cloning of a novel human gene encoding a 63-kDa protein and its sublocalization within the 11q13 locus. Genomics 41, 397-405.
16. Potterton, L., McNicholas, S., Krissinel, E., Gruber, J., Cowtan, K., Emsley, P., Murshudov, G. N., Cohen, S., Perrakis, A., and Noble, M. (2004). Developments in the CCP4 molecular-graphics project. Acta Crystallogr D Biol Crystallogr 60, 2288-2294.
17. Takahashi, Y., Coppola, D., Matsushita, N., Cualing, H. D., Sun, M., Satoe, Y., Liang, C., Jung, J. U., Cheng, J. Q., Mule, J. J., et al. (2007). Bif-1 interacts with Beclin 1 through UVRAG and regulates autophagy and tumorigenesis. Nat Cell Biol 9, 1142-1151.
18. Takahashi, Y., Meyerkord, C. L., and Wang, H. G. (2009). Bif-1/endophilin B1: a candidate for crescent driving force in autophagy. Cell Death Differ 16, 947-955.
19. Terwilliger, T C., and Berendzen, J. (1999). Automated MAD and MIR structure solution. Acta Crystallogr D Biol Crystallogr 55, 849-861.
20. Zhao, Z., Oh, S., Li, D., Ni, D., Pirooz, S. D., Lee, J. H., Yang, S., Lee, J. Y, Ghozalli, I., Costanzo, V., et al. (2012). A dual role for UVRAG in maintaining chromosomal stability independent of autophagy. Dev Cell 22, 1001-1016.
21. Zhong, Y, Wang, Q. J., Li, X., Yan, Y., Backer, J. M., Chait, B. T., Heintz, N., and Yue, Z. (2009). Distinct regulation of autophagic activity by Atg14L and Rubicon associated with Beclin 1-phosphatidylinositol-3-kinase complex. Nat Cell Biol 11, 468-476.
22. U.S. Pat. No. 8,802,633, Autophagy-inducing peptide analogs, Published on Aug. 12, 2014.
23. (2002). “High-throughput structure determination. Proceedings of the 2002 CCP4 (Collaborative Computational Project in Macromolecular Crystallography) study weekend. January, 2002. York, United Kingdom.” Acta Crystallogr D Biol Crystallogr 58(Pt 11): 1897-1970.
24. Bricogne, G., et al. (2003). “Generation, representation and flow of phase information in structure determination: recent developments in and around SHARP 2.0.” Acta Crystallogr D Biol Crystallogr 59(Pt 11): 2023-2030.
25. Emsley, P. and K. Cowtan (2004). “Coot: model-building tools for molecular graphics.” Acta Crystallogr D Biol Crystallogr 60(Pt 12 Pt 1): 2126-2132.
26. Kim. Y. W., et al. (2011). “Synthesis of all-hydrocarbon stapled alpha-helical peptides by ring-closing olefin metathesis.” Nat Protoc 6(6): 761-771.
27. Li, X., et al. (2012). “Imperfect interface of Beclin1 coiled-coil domain regulates homodimer and heterodimer formation with Atg14L and UVRAG.” Nat Commun 3: 662.
28. Li. X., et al. (2012). “Imperfect interface of Beclin1 coiled-coil domain regulates homodimer and heterodimer formation with Atg14L and UVRAG.” NATURE COMMUNICATIONS 3: 662.
29. Li, X., et al. (2012). “The BECN1 coiled coil domain: an “imperfect” homodimer interface that facilitates ATGI4 and UVRAG binding.” Autophagy 8(8): 1258-1260.
30. Liang, C., et al. (2006). “Autophagic and tumour suppressor activity of a novel Beclin1-binding protein UVRAG.” Nat Cell Biol 8(7): 688-699.
31. Liang, C., et al. (2007). “UVRAG: a new player in autophagy and tumor cell growth.” Autophagy 3(1): 69-71.
32. Liang, C., et al. (2008). “Beclin1-binding UVRAG targets the class C Vps complex to coordinate autophagosome maturation and endocytic trafficking.” Nat Cell Biol 10(7): 776-787.
33. Liang, C., et al. (2008). “Beyond autophagy: the role of UVRAG in membrane trafficking.” Autophagy 4(6): 817-820.
34. Matsunaga, K., et al. (2009). “Two Beclin 1-binding proteins, Atg14L and Rubicon, reciprocally regulate autophagy at different stages.” Nat Cell Biol 11(4): 385-396.
35. McKnight, N. C., et al. (2014). “Beclin 1 is required for neuron viability and regulates endosome pathways via the UVRAG-VPS34 complex.” PLoS Genet 10(10): e1004626.
36. Murshudov, G. N., et al. (1997). “Refinement of macromolecular structures by the maximum-likelihood method.” Acta Crystallogr D Biol Crystallogr 53(Pt 3): 240-255.
37. Otwinowski, Z. and W. Minor (1997). “Processing of X-ray diffraction data collected in oscillation mode.” Methods Enzymol 276: 307-326.
38. Perelman, B., et al. (1997). “Molecular cloning of a novel human gene encoding a 63-kDa protein and its sublocalization within the 11q13 locus.” Genomics 41(3): 397-405.
39. Potterton, L., et al. (2004). “Developments in the CCP4 molecular-graphics project.” Acta Crystallogr D Biol Crystallogr 60(Pt 12 Pt 1): 2288-2294.
40. Rostislavleva, K., et al. (2015). “Structure and flexibility of the endosomal Vps34 complex reveals the basis of its function on membranes.” Science 350(6257): aac7365.
41. Takahashi, Y. et al. (2007). “Bif-1 interacts with Beclin 1 through UVRAG and regulates autophagy and tumorigenesis.” Nat Cell Biol 9(10): 1142-1151.
42. Takahashi, Y, et al. (2009). “Bif-1/endophilin B1: a candidate for crescent driving force in autophagy.” Cell Death Differ 16(7): 947-955.
43. Terwilliger, T. C. and J. Berendzen (1999). “Automated MAD and MIR structure solution.” Acta Crystallogr D Biol Crystallogr 55(Pt 4): 849-861.
44. Zhao, Z., et al. (2012). “A dual role for UVRAG in maintaining chromosomal stability independent of autophagy.” Dev Cell 22(5): 1001-1016.
45. Zhong, Y., et al. (2009). “Distinct regulation of autophagic activity by Atg14L and Rubicon associated with Beclin 1-phosphatidylinositol-3-kinase complex.” Nat Cell Biol 11(4): 468-476.

What is claimed is:

1. A hydrocarbon-stapled polypeptide designed to target a polypeptide comprising amino acid residues 231-245 of rat Beclin 1 (SEQ ID No.: 15), or amino acids 233-247 of human Beclin 1 (SEQ ID No.: 16), said hydrocarbon-stapled polypeptide comprises an amino acid sequence that is at least 85% identical to amino acid residues 191-205 of rat Beclin 1 (SEQ ID No.: 17), or amino acids 193-207 of human Beclin 1 (SEQ ID No.: 18).

2. The hydrocarbon-stapled polypeptide of claim 1, wherein said hydrocarbon-stapled polypeptide is about 10-40 amino acids in length.

3. The hydrocarbon-stapled polypeptide of claim 1, wherein said hydrocarbon-stapled polypeptide comprises one or more α,α-disubstituted 5-carbon olefinic amino acids.

4. The hydrocarbon-stapled polypeptide of claim 1, wherein said hydrocarbon-stapled polypeptide comprises one or more α,α-disubstituted 8-carbon olefinic amino acids.

5. The hydrocarbon-stapled polypeptide of claim 1, wherein said hydrocarbon-stapled polypeptide has an affinity for said polypeptide comprising amino acid residues 231-245 of rat Beclin 1 (SEQ ID No.: 15), or amino acids 233-247 of human Beclin 1 (SEQ ID No.: 16), of at least 2 μM.

6. The hydrocarbon-stapled polypeptide of claim 1, wherein said hydrocarbon-stapled polypeptide has the sequence of one of SEQ ID NO. 1-12.

7. A pharmaceutical composition comprising the hydrocarbon-stapled polypeptide of claim 1.

8. The pharmaceutical composition of claim 7, further comprising one or more pharmaceutically acceptable excipients, vehicles or carriers.

9. The pharmaceutical composition of claim 7, wherein said pharmaceutical composition is formulated in the form of a cream, gel, ointment, suppository, tablet, granule, injection, powder, solution, suspension, spray, patch or capsule.

10. A method of enhancing autophagy or endocytic trafficking, comprising the step of contacting a population of cells with the hydrocarbon-stapled polypeptide of claim 1, thereby enhancing lysosomal degradation of one or more target proteins.

11. The method of claim 10, wherein the target protein is EGFR.

12. The method of claim 11, wherein the cells treated with said hydrocarbon-stapled polypeptide have decreased EGFR-driven cell proliferation.

13. A method of inhibiting cancer cell growth, comprising administering an effective amount of the hydrocarbon-stapled polypeptide of claim 1 to a subject in need thereof.

14. The method of claim 13, wherein the subject is a vertebrate, a mammal or human.

15. The method of claim 13, wherein the cancer cell growth comprises EGFR-driven cell proliferation.

16. The method of claim 13, wherein the cancer cells are non-small cell lung cancer cells, breast cancer cells, colon cancer cells, ovarian cancer cells, carcinoma cells, sarcoma cells, lung cancer cells, fibrosarcoma cells, myosarcoma cells, liposarcoma cells, chondrosarcoma cells, osteogenic sarcoma cells, chordoma cells, angiosarcoma cells, endotheliosarcoma cells, lymphangiosarcoma cells, lymphangioendotheliosarcoma cells, synovioma cells, mesothelioma cells, Ewing's tumor cells, leiomyosarcoma cells, rhabdomyosarcoma cells, gastric cancer cells, esophageal cancer cells, rectal cancer cells, pancreatic cancer cells, prostate cancer cells, uterine cancer cells, head and neck cancer cells, skin cancer cells, brain cancer cells, squamous cell carcinoma, sebaceous gland carcinoma cells, papillary carcinoma cells, papillary adenocarcinoma cells, cystadenocarcinoma cells, medullary carcinoma cells, bronchogenic carcinoma cells, renal cell carcinoma cells, hepatoma cells, bile duct carcinoma cells, choriocarcinoma cells, seminoma cells, embryonal carcinoma cells, Wilm's tumor cells, cervical cancer cells, testicular cancer cells, small cell lung carcinoma cells, bladder carcinoma cells, epithelial carcinoma cells, glioma cells, astrocytoma cells, medulloblastoma cells, craniopharyngioma cells, ependymoma cells, pinealoma cells, hemangioblastoma cells, acoustic neuroma cells, oligodendroglioma cells, meningioma cells, melanoma cells, neuroblastoma cells, retinoblastoma cells, T-cells or natural killer cells of leukemia, lymphoma cells, or Kaposi's sarcoma cells.


SEQUENCE LISTING (33 sequences)

SEQ ID NO: 1 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLIQELXDREAQRXV

SEQ ID NO: 2 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLIQELXDREAQRXS

SEQ ID NO: 3 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLISELXDREKQRXV

SEQ ID NO: 4 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLISELXDREKQRXA

SEQ ID NO: 5 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLIQELXDREKQRXS

SEQ ID NO: 6 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLISELXDREKQRXS

SEQ ID NO: 7 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLIQELXDREKQRXR

SEQ ID NO: 8 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLIQELXDREKERXA

SEQ ID NO: 9 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
LLISELXDREKQRXA

SEQ ID NO: 10 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
RLLSELXDREKQRXA

SEQ ID NO: 11 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
LLLSRLXDREKQRXA

SEQ ID NO: 12 - protein, 15 aa, Artificial Sequence
  Synthetic polypeptide
  MISC_FEATURE (7)..(7): Xaa = (R)-2-amino-2-methyldec-9-enoic acid residue
  MISC_FEATURE (14)..(14): Xaa = (S)-2-amino-2-methylhept-6-enoic acid residue
LLISQLXDREKQRXA

SEQ ID NO: 13 - protein, 11 aa, Artificial Sequence
  Synthetic polypeptide
YGRKKRRQRRR

SEQ ID NO: 14 - protein, 13 aa, Artificial Sequence
  Synthetic polypeptide
RALRIQSKEELRD

SEQ ID NO: 15 - protein, 15 aa, Unknown
  Beclin 1
YSEFKRQQLELDDEL

SEQ ID NO: 16 - protein, 15 aa, Unknown
  Human Beclin 1
YSEFKRQQLELDDEL

SEQ ID NO: 17 - protein, 15 aa, Unknown
  Rat Beclin 1
RLIQELEDVEKNRKV

SEQ ID NO: 18 - protein, 15 aa, Unknown
  Human Beclin 1
RLIQELEDVEKNRKI

SEQ ID NO: 19 - protein, 50 aa, Unknown
  Beclin 1
DSEQAQRELKELALEEERLIQELEDVEKNRKVVAENLEKVQAEAERLDQE

SEQ ID NO: 20 - protein, 49 aa, Unknown
  Beclin 1
EERLIQELEDVEKNRKVVAENLEKVQAEAERLDQEEAQYQREYSEFKRQ

SEQ ID NO: 21 - protein, 49 aa, Unknown
  Beclin 1
RKVVAENLEKVQAEAERLDQEEAQYQREYSEFKRQQLELDDELKSVENQ

SEQ ID NO: 22 - protein, 50 aa, Unknown
  Beclin 1
AERLDQEEAQYQREYSEFKRQQLELDDELKSVENQMRYAQMQADKLKKTN

SEQ ID NO: 23 - protein, 93 aa, Unknown
  Beclin 1
DSEQLQRELKELALEEERLIQELEDVEKNRKVVAENLEKVQAEAERLDQEEAQYQREYSEFKRQQLELDDELKSVENQMRYAQMQLDKLKKTN

SEQ ID NO: 24 - protein, 48 aa, Unknown
  UVRAG
TSNELKKESESLRLKILVLRNELERQKKALGREVAFLHKQQMALQDKG

SEQ ID NO: 25 - protein, 117 aa, Unknown
  Atg4L
MDYKDDDDKKQEEFQKEVLKAMEGKRLTDQLRWKIMSCKMRIEQLKQTICKGNEEMKKNSEGLLKNKEKNQKLYSRAQRHQEKKEKIQRHNRKLGDLVEKKTIDLKSHYERLARLRR

SEQ ID NO: 26 - protein, 108 aa, Artificial Sequence
  Synthetic polypeptide
DSEQAQRELKELALEEERLIQELEDVEKNRKVVAENLEKVQAEAERLDQEGSGSGSGSGSTSNELKKESESLRLKILVLRNELERQKKALGREVAFLHKQQMALQDKG

SEQ ID NO: 27 - protein, 71 aa, Unknown
  UVRAG
TSNELKKESESLRLKILVLRNELERQKKALGREVAFLHKQQMALQDKGSAFSTEHGKLQLQKDSLSELRKE

SEQ ID NO: 28 - protein, 698 aa, Unknown
  UVRAG
MSSCASLGGPVPLPPPGPSAALTSGAPARALHVELPSQQRRLRHLRNIAARNIVNRNGHQLLDTYFTLHLCDNEKIFKEFYRSEVIKNSLNPTWRSLDFGIMPDRLDTSVSCFVVKIWGGKEEAFQLLIEWKVYLDGLKYLGQQIHARNQNEIIFGLNDGYYGAPCEHKGHPNAQKNLLQVDQNCVRNSYDVFSLLRLHRAQCAIKQTQVTVQRLGKEIEEKLRLTSTSNELKKESECLRLKILVLRNELERQKKALGREVAFLHKQQMALQDKGSAFSTEHGKLQLQKDSLSELRKECTAKRELFLKTNAQLTIRCRQLLSELSYIYPIDLNEHKDYFVCGVKLPNSEDFQAKEDGSIAVALGYTAHLVSMISFFLQVPLRYPIIHKGSRSTIKDNINDKLTEKEREFPLYPKGGEKLQFDYGVYLLNKNIAQLRYQHGLGTPDLRQTLPNLKNFMEHGLMVRCDRHHISNAIPVPKRQSSTFGGADGGFSAGIPSPDKVHRKRASSENERLQYKTPPPSYNSALTQPGVAMPTSGDSERKVAPLSSSLDTSLDFSKENKKAGVDLGSSVSGDHGNSDSGQEQGEALPGHLAAVNGTALPSEQAGPAGTLLPGSCHPAPSAELCCAVEQAEEIIGLEATGFTSGDQLEALSCIPVDSAVAVECDEQVLGEFEEFSRRIYALSENVSSFRRPRRSSDK

SEQ ID NO: 29 - protein, 698 aa, Artificial Sequence
  Mutant of UVRAG
MSSCASLGGPVPLPPPGPSAALTSGAPARALHVELPSQQRRLRHLRNIAARNIVNRNGHQLLDTYFTLHLCDNEKIFKEFYRSEVIKNSLNPTWRSLDFGIMPDRLDTSVSCFVVKIWGGKEEAFQLLIEWKVYLDGLKYLGQQIHARNQNEIIFGLNDGYYGAPCEHKGHPNAQKNLLQVDQNCVRNSYDVFSLLRLHRAQCAIKQTQVTVQRLGKEIEEKLRLTSTSNELKKESECLRLKILVERNELERQKKALGREVAFLHKQQMALQDKGSAFSTEHGKLQLQKDSLSELRKECTAKRELFLKTNAQLTIRCRQLLSELSYIYPIDLNEHKDYFVCGVKLPNSEDFQAKEDGSIAVALGYTAHLVSMISFFLQVPLRYPIIHKGSRSTIKDNINDKLTEKEREFPLYPKGGEKLQFDYGVYLLNKNIAQLRYQHGLGTPDLRQTLPNLKNFMEHGLMVRCDRHHISNAIPVPKRQSSTFGGADGGFSAGIPSPDKVHRKRASSENERLQYKTPPPSYNSALTQPGVAMPTSGDSERKVAPLSSSLDTSLDFSKENKKAGVDLGSSVSGDHGNSDSGQEQGEALPGHLAAVNGTALPSEQAGPAGTLLPGSCHPAPSAELCCAVEQAEEIIGLEATGFTSGDQLEALSCIPVDSAVAVECDEQVLGEFEEFSRRIYALSENVSSFRRPRRSSDK

SEQ ID NO: 30 - protein, 698 aa, Artificial Sequence
  Mutant of UVRAG
MSSCASLGGPVPLPPPGPSAALTSGAPARALHVELPSQQRRLRHLRNIAARNIVNRNGHQLLDTYFTLHLCDNEKIFKEFYRSEVIKNSLNPTWRSLDFGIMPDRLDTSVSCFVVKIWGGKEEAFQLLIEWKVYLDGLKYLGQQIHARNQNEIIFGLNDGYYGAPCEHKGHPNAQKNLLQVDQNCVRNSYDVFSLLRLHRAQCAIKQTQVTVQRLGKEIEEKLRLTSTSNELKKESECLRLKILVERNEEERQKKALGREVAFLHKQQMALQDKGSAFSTEHGKLQLQKDSLSELRKECTAKRELFLKTNAQLTIRCRQLLSELSYIYPIDLNEHKDYFVCGVKLPNSEDFQAKEDGSIAVALGYTAHLVSMISFFLQVPLRYPIIHKGSRSTIKDNINDKLTEKEREFPLYPKGGEKLQFDYGVYLLNKNIAQLRYQHGLGTPDLRQTLPNLKNFMEHGLMVRCDRHHISNAIPVPKRQSSTFGGADGGFSAGIPSPDKVHRKRASSENERLQYKTPPPSYNSALTQPGVAMPTSGDSERKVAPLSSSLDTSLDFSKENKKAGVDLGSSVSGDHGNSDSGQEQGEALPGHLAAVNGTALPSEQAGPAGTLLPGSCHPAPSAELCCAVEQAEEIIGLEATGFTSGDQLEALSCIPVDSAVAVECDEQVLGEFEEFSRRIYALSENVSSFRRPRRSSDK

SEQ ID NO: 31 - protein, 698 aa, Artificial Sequence
  Mutant of UVRAG
MSSCASLGGPVPLPPPGPSAALTSGAPARALHVELPSQQRRLRHLRNIAARNIVNRNGHQLLDTYFTLHLCDNEKIFKEFYRSEVIKNSLNPTWRSLDFGIMPDRLDTSVSCFVVKIWGGKEEAFQLLIEWKVYLDGLKYLGQQIHARNQNEIIFGLNDGYYGAPCEHKGHPNAQKNLLQVDQNCVRNSYDVFSLLRLHRAQCAIKQTQVTVQRLGKEIEEKLRLTSTSNEEKKESECERLKILVERNEEERQKKALGREVAFEHKQQMALQDKGSAFSTEHGKLQLQKDSLSELRKECTAKRELFLKTNAQLTIRCRQLLSELSYIYPIDLNEHKDYFVCGVKLPNSEDFQAKEDGSIAVALGYTAHLVSMISFFLQVPLRYPIIHKGSRSTIKDNINDKLTEKEREFPLYPKGGEKLQFDYGVYLLNKNIAQLRYQHGLGTPDLRQTLPNLKNFMEHGLMVRCDRHHISNAIPVPKRQSSTFGGADGGFSAGIPSPDKVHRKRASSENERLQYKTPPPSYNSALTQPGVAMPTSGDSERKVAPLSSSLDTSLDFSKENKKAGVDLGSSVSGDHGNSDSGQEQGEALPGHLAAVNGTALPSEQAGPAGTLLPGSCHPAPSAELCCAVEQAEEIIGLEATGFTSGDQLEALSCIPVDSAVAVECDEQVLGEFEEFSRRIYALSENVSSFRRPRRSSDK

SEQ ID NO: 32 - protein, 698 aa, Artificial Sequence
  Mutant of UVRAG
MSSCASLGGPVPLPPPGPSAALTSGAPARALHVELPSQQRRLRHLRNIAARNIVNRNGHQLLDTYFTLHLCDNEKIFKEFYRSEVIKNSLNPTWRSLDFGIMPDRLDTSVSCFVVKIWGGKEEAFQLLIEWKVYLDGLKYLGQQIHARNQNEIIFGLNDGYYGAPCEHKGHPNAQKNLLQVDQNCVRNSYDVFSLLRLHRAQCAIKQTQVTVQRLGKEIEEKLRLTSTSNEEKKESECERLKILVERNEEERQKKALGREVAFEHKQQMAEQDKGSAFSTEHGKLQLQKDSLSELRKECTAKRELFLKTNAQLTIRCRQLLSELSYIYPIDLNEHKDYFVCGVKLPNSEDFQAKEDGSIAVALGYTAHLVSMISFFLQVPLRYPIIHKGSRSTIKDNINDKLTEKEREFPLYPKGGEKLQFDYGVYLLNKNIAQLRYQHGLGTPDLRQTLPNLKNFMEHGLMVRCDRHHISNAIPVPKRQSSTFGGADGGFSAGIPSPDKVHRKRASSENERLQYKTPPPSYNSALTQPGVAMPTSGDSERKVAPLSSSLDTSLDFSKENKKAGVDLGSSVSGDHGNSDSGQEQGEALPGHLAAVNGTALPSEQAGPAGTLLPGSCHPAPSAELCCAVEQAEEIIGLEATGFTSGDQLEALSCIPVDSAVAVECDEQVLGEFEEFSRRIYALSENVSSFRRPRRSSDK

SEQ ID NO: 33 - protein, 93 aa, Artificial Sequence
  Synthetic polypeptide
DSEQLQREAKELALEEERLIQELEDVEKNRKVVAENLEKVQAEAERLDQEEAQYQREYSEFKRQQLELDDELKSVENQMRYAQMQLDKLKKTN